SEQUENCE LISTING (9 sequences)

SEQ ID NO: 1 - protein, 8 aa, Artificial Sequence
  Description of Artificial Sequence human peptide
CYSGDENP

SEQ ID NO: 2 - protein, 8 aa, Artificial Sequence
  Description of Artificial Sequence human peptide
LQFGDIPT

SEQ ID NO: 3 - protein, 9 aa, Artificial Sequence
  Description of Artificial Sequence human peptide
QLLQDTPVA

SEQ ID NO: 4 - protein, 8 aa, Artificial Sequence
  Description of Artificial Sequence human peptide
KVWQDACS

SEQ ID NO: 5 - protein, 8 aa, Artificial Sequence
  Description of Artificial Sequence human peptide
QFAVDMQT

SEQ ID NO: 6 - protein, 14 aa, Artificial Sequence
  Description of Artificial Sequence human peptide
NCEHGAFSCPACYN

SEQ ID NO: 7 - protein, 14 aa, Artificial Sequence
  Description of Artificial Sequence rat peptide
RTAAALTSCPACYN

SEQ ID NO: 8 - protein, 14 aa, Artificial Sequence
  Description of Artificial Sequence human peptide
GDCYSGDENPDTEC

SEQ ID NO: 9 - protein, 14 aa, Artificial Sequence
  Description of Artificial Sequence human peptide
GALQFGDIPTSHLL

(19) United States
(12) Patent Application Publication
(10) Pub. No.: US 2002/0076736 A1
(43) Pub. Date: Jun. 20, 2002

(54) Methods of affecting laminin 5 processing

(76) Inventors: Paul R. Findell, San Francisco, CA (US); M. Peter Marinkovich III, Redwood City, CA (US)

Correspondence Address: FIBROGEN, INC., Intellectual Property Department, 225 Gateway Blvd., South San Francisco, CA, 94080, US

(21) Appl. No.: 09/854,188

(22) Filed: May 11, 2001

Related U.S. Application Data
(60) Provisional application No. 60/203,708, filed on May 12, 2000.

(51) Int. Cl. G01N 33/574; C12N 9/64; A61K 31/19; A61K 31/4439

(52) U.S. Cl. 435/7.23; 435/226; 514/341; 514/575

(57) ABSTRACT

The present invention relates to a method for diagnosing, treating, and preventing conditions associated with altered expression or activity of laminin 5 using BMP-1 inhibitors.

### CROSS-REFERENCE TO RELATED APPLICATION

This application is related to provisional patent application Ser. No. 60/203,708, filed May 12, 2000, from which priority is claimed under 35 USC § 119(e)(1) and which is incorporated herein by reference in its entirety.

### BACKGROUND OF THE INVENTION

Squamous cell carcinoma (SCC) is a malignant neoplasm of epithelial cells, e.g., keratinocytes, bronchial epithelia, etc. Squamous cell carcinomas (SCCs) can include, for example, skin, oral, lung, cervical, and colorectal carcinomas. Squamous cell carcinoma in the form of skin cancer is often the result of long-term sun damage to the skin. SCCs of the skin are most common on the face, as the face typically receives the most exposure to the sun and other environmental hazards. Thus, the skin, lips, mouth, throat, esophagus, etc., are often affected.

SCC is a common form of cancer and is the second most common form of skin cancer in the U.S., representing about 100,000 cases of skin cancer per year. SCC in the form of lung bronchogenic carcinomas appears in the U.S. in about 60,000 cases per year, representing thirty percent of all lung bronchogenic carcinomas diagnosed yearly in the U.S. SCC is a major form of oral, head, and neck cancers, with SCC-associated cases numbering 40,000 per year in the U.S. In addition, ninety percent of all cervical malignancies are associated with SCC; such cases number 15,000 cases per year in the U.S. and over 430,000 cases per year worldwide, with over 200,000 deaths per year. Disseminated squamous cell carcinoma is the most common cause of death in patients with recessive dystrophic epidermolysis bullosa who survive into adulthood. (Fine et al. (1991) J. Am. Acad. Dermatol. 24:119-135; Marinkovich et al. (1999) “Inherited epidermolysis bullosa,” in Dermatology in General Medicine, Freedberg et al., eds., McGraw-Hill, N.Y.) SCC also can complicate dominant dystrophic epidermolysis bullosa. (Christiano et al (1999) Exp. Dermatol. 8:146-152.)

SCCs are highly invasive and metastatic. Squamous cell carcinomas can invade neighboring tissues, such as the eye, mouth, and lymph nodes, and can also metastasize to more distant locations of the body, including internal organs and tissues. Thirty percent of all head and neck SCCs metastasize. Additional carcinomas sometimes develop in sites close to a first-detected carcinoma, as these surrounding regions were exposed to the same carcinoma-inducing effect. Surgical resection is a common form of treatment. However, SCCs are associated with a comparatively high risk of recurrence, resulting in significant mortality. Fifty percent of patients with metastatic esophageal cancer die within two years of tumor resection.

SCC can be diagnosed by biopsy. Squamous cell carcinomas are typically not as distinct as basal cell carcinomas or melanomas, making detection and diagnosis difficult in some circumstances. The detection of SCC can sometimes be difficult due to slow manifestation of detectable symptoms. In certain sites, such as the throat, development of SCC can lead to local symptoms such as pain and difficulty in swallowing, and hoarseness. However, diagnosis is often delayed as local symptoms or pain resulting from nerve involvement might not occur until development of a large primary tumor. The initial manifestation can consist of regional nodal metastases. Distant metastases will occur after primary local development or nodal involvement.

Current methods of treatment include surgery, radiotherapy, and chemotherapy. There are a variety of different treatment options for removal of squamous cell carcinomas and the technique used for each case will differ depending on the characteristics of the individual lesion. An appropriate treatment regime is determined by the stage of the disease, its specific histopathologic features, and the medical history of an individual patient, such as whether the patient is undergoing prior treatment such as chemotherapy, etc.

Factors such as shape, size, and tumor location are all important elements. Surgical excision is the preferred option for removal in most cases where involvement appears to be only local. Such procedures can often be performed as outpatient procedures. Other forms of treatment used in some instances include electrodessication (burning lesions away using an electric spark), as well as x-ray radiation and curettage (scraping the abnormal tissue off the skin). Another form of treatment may include local radiotherapy to the affected area of the skin.

Surgery and radiotherapy provide similar survival rates when employed in treatment of early disease. For treatment of more locally advanced disease, surgery and radiotherapy, pre-operative or post-operative, are often used in combination. Chemotherapy has been incorporated into initial treatment plans for patients with locally advanced disease in order to improve the disease-free survival rate for such patients beyond that for when only conventional surgery, or radiotherapy, or both, are used.

Laminin 5 is a heterotrimeric extracellular matrix (ECM) protein that promotes cell migration and cell scattering, and tumor invasion. Laminin 5 is initially synthesized and secreted in an unprocessed form with an α3 chain of 200 kDa, a β3 chain of 140 kDa, and a γ2 chain of 155 kDa. (Marinkovich et al (1992) J. Biol. Chem. 267:17900-17906.) The human α3 chain of laminin 5 has been cloned. (Ryan et al., (1994) J. Biol. Chem. 269:22779-22787; GenBank Accession No. NM_{—}000227 (polynucleotide) and Genbank Accession No. NP_{—}000218 (polypeptide).) The human β3 chain of laminin 5 has been cloned. (GenBank Accession No. D37766 (polynucleotide) and GenBank Accession No. BAA22263 (polypeptide).) Two variants of the human γ2 chain of laminin 5 have been cloned. (Airenne et al., (1996) Genomics 32:54-64; variant 1 of human γ2 chain of laminin 5, GenBank Accession No. NM_{—}005562 (polynucleotide) and GenBank Accession No. NP_{—}005553 (polypeptide); variant 2 of human γ2 chain of laminin 5, GenBank Accession No. NM_{—}018891 (polynucleotide) and GenBank Accession No. NP_{—}061486 (polypeptide).)

Following secretion, laminin 5 undergoes several processing steps. Laminin 5 is a heterotrimeric basement membrane zone protein vital to the structural stability of the skin. It functions as an adhesive molecule by forming a bridge between other components of the extracellular matrix (ECM) and the integrin receptors of the basal keratinocytes. During processes such as wound healing and tumor invasion, however, keratinocytes are required to become highly migratory and the adhesive properties of laminin 5 are altered by proteolytic processing. The 200 kDa α3 chain is processed to a 165 kDa product, and the 155 kDa γ2 chain is processed to a 105 kDa product. Processing of the α3 chains of laminin 5 stimulates the cell migration and scattering activities of laminin 5.

SCC tumors deposit large amounts of laminin 5 at their leading edges. This deposition of laminin 5 is believed to serve as a substrate for tumor invasion. (See, e.g., Pyke et al. (1995) Canc. Res. 55:4132-4139; Berndt et al. (1997) Invasion and Metastasis 17:251-258.) Increased laminin 5 immunoreactivity has been found indicative of a poor prognosis in patients with squamous cell carcinoma of the tongue. (Ono et al. (1999) Cancer 85:2315-2321.) Other conditions are associated with increased expression or activity of laminin 5. Laminin 5 is preferentially expressed by invading malignant cells of many human carcinomas in addition to squamous cell carcinomas, colon and mammary carcinomas (Pyke et al., Am. J. Pathol. 1994, 145(4):782-91), and malignant gliomas (Fukushima et al., Int. J. Cancer 1998, 76: 63-72).

Various processing steps of laminin 5 have been shown to influence cell migration. One laminin 5 processing step occurs on the α3 chain in the G4 domain and affects both cell migration and hemidesmosome formation. (Goldfinger et al. (1998) J. Cell Bio. 141:255-265.) A second processing step mediated by MMP-2 takes place at the junction of domains II and III in the γ2 chain and causes increased migration of a variety of cell types. (Gianelli et al (1997) Science 277:225-228.) This processing takes place in rat tissues, but has not been shown to occur in human tissues. A third processing event involves cleavage of the γ2 chain near the junction of domains III and IV by bone morphogenetic protein-1 (BMP-1) and BMP-1 related proteins. (Amano et al. (2000) J. Biol. Chem. 275:22728-22735.) However, the mechanism by which laminin 5 affects SCC tumor invasiveness has remained unclear.

The BMP-1 and mammalian tolloid (mTld) proteins are alternatively spliced gene products belonging to a subfamily of astacin-like proteases. (Finelli et al. (1995) Genetics 141:271-281.) BMP-1 has been identified as possessing BMP-1 activity, as have mTld and other BMP-1 related proteins, including mammalian tolloid-like-1 (mTll-1) and mammalian tolloid-like-2 (mTll-2). (Li et al. (1996) PNAS 93:5127-5130; Kessler et al. (1996) Science 271:360-362; Takahara et al. (1996) Genomics 34: 157-165; Scott et al. (1999) Dev. Biol. 213:283-300; Takahara et al. (1994) J. Biol. Chem. 269:32572-32578.) BMP-1 and mTld have similar substrate specificity and both demonstrate C-proteinase activity with respect to type I, II, and III procollagens. (Kessler et al. (1996), supra.) Such cleavage sequences can also be found in other proteins, for example, chordin, and lysyl oxidase, and such proteins thus also serve as substrates for the processing by the same enzymatic activity. The mTld variant is produced in keratinocytes. (Lee et al. (1997) J. Bio. Chem. 272:19059-19066.)

In summary, SCC is a common and a dangerous form of cancer, associated with a high risk of metastasis, and can lead to serious impairment or loss of function, depending on the organ affected and the extent of the cancerous invasion. Current methods of treatment require continued monitoring in view of the metastatic nature of the disease and the risk of nodal involvement. Therefore, there is a need for methods of early stage and reliable methods of detection of SCC, and methods for quick and effective treatment of SCC upon detection. There is a need for treatment of other conditions associated with increased laminin 5 expression and activity, such as glioma and other cancers. Treatments for continued prevention after detection and removal of SCCs and other malignancies are also desired.

Before the present invention, the mechanism by which laminin 5 affects cell migration and tumor invasion in cancers such as SCCs, gliomas, and other conditions associated with increased expression or activity of laminin 5 remained unclear. An understanding of the mechanism of laminin processing could be valuable in establishing means of altering laminin 5 processing to counteract the role of laminin 5 in development of such conditions and disorders. There is thus a need for characterization of the proteins which process laminin 5 and the role of these proteins in cell migration. The present invention is associated with the discovery that processing of human laminin 5 by BMP-1, mTld, and other BMP-1 related proteins is of crucial importance in cell migration and scattering, and tumor invasion, and provides methods of diagnosing, preventing, and treating cancers such as SCCs, gliomas, and other conditions associated with increased expression or activity of laminin 5.

### SUMMARY OF THE INVENTION

The present invention relates to the discovery that BMP-1 related proteins are involved in the processing of laminin 5. The present invention further relates to the discovery that inhibition of processing of laminin 5 by BMP-1 related proteins can inhibit cell migration and invasion in conditions associated with increased expression or activity of laminin 5, including cancers such as SCCs, gliomas, etc. Therefore, in one embodiment, the present invention provides a method of regulating laminin 5 expression or activity, the method comprising contacting laminin 5 with an effective amount of an agent that affects processing of laminin 5 by a BMP-1 related protein. In further aspects, the BMP-1 related protein is selected from the group consisting of BMP-1, mTld, mTll-1, and mTll-2. In other aspects, the agent can be an antibody that binds to a BMP-1 related protein, or an antibody that binds to laminin 5 and prevents processing of laminin 5 by a BMP-1 related protein.

In one aspect, the invention provides a method of treating a condition characterized by increased expression or activity of laminin 5, the method comprising administering to a subject in need an effective amount of an agent that affects processing of laminin 5 by a BMP-1 related protein. In one aspect, the present invention provides a method of treating cancer, the method comprising administering to a subject in need an effective amount of an agent that affects processing of laminin 5 by a BMP-1 related protein. In other embodiments, methods of treating squamous cell carcinoma, the methods comprising administering to a subject in need an effective amount of an agent that affects processing of laminin 5 by a BMP-1 related protein, are provided. Methods of treating glioma are also provided.

In one embodiment, the present invention relates to a method of treating a condition characterized by a neoplasm of epithelial cells or epithelia, the method comprising administering to a subject in need an effective amount of an agent that affects laminin 5 processing by BMP-related proteins. In another embodiment, the invention provides a method of treating squamous cell carcinoma, the method comprising administering to a subject in need an effective amount of an agent that affects laminin 5 processing by BMP-related proteins. In further embodiments, the squamous cell carcinoma is selected from the group consisting of skin cancer, lung cancer, head cancer, neck cancer, oral cancer, cervical cancer, gastric cancer, esophageal cancer, etc. The SCC can be sun-induced, i.e., actinically derived SCC, or can result from transplant or invasive surgery, or follow other immunosuppressive situations. In other embodiments, the SCC is derived from chronic inflammation, Marjolin's ulcer, burns or scars, etc. The SCC can be virally induced, or can be adenoid (acantholytic) SCC, spindle cell SCC, verrucous carcinoma (VC), keratoacanthoma (KA), nodular SCC, periungual SCC, or any other epithelial carcinomas.

In other embodiments, the agent affects the processing of the α3 chain of laminin 5, or the processing of the γ2 chain of laminin 5. In further aspects, the agent affects processing of laminin 5 by BMP-1, by mTld, by mTll-1, or by mTll-2.

The present invention additionally provides for a method of treatment wherein the administration of the agent takes place at the time of or subsequent to surgical resection and the agent additionally confers an anti-scarring effect. Compositions for the treatment of conditions associated with increased expression or activity of laminin 5, including cancers such as squamous cell carcinoma and glioma, are also provided, the compositions comprising an agent that affects processing of laminin 5 by a BMP-1 related protein and an acceptable carrier. In additional embodiments, the agent affects the processing of laminin 5 by at least one of the proteins selected from the group consisting of BMP-1, mTld, mTll-1, and mTll-2. In further aspects, the agent is selected from the group consisting of a hydroxamate, hydroxamic acid, or derivative thereof. In specific embodiments, the agent is Compound 1, 2, or 3, as described, infra.

In additional embodiments, the present invention provides methods of diagnosing the presence of a conditions characterized by increased expression or activity of laminin 5 in a subject, the methods comprising: (a) obtaining a sample; (b) detecting the level of expression or activity of a BMP-1 related protein in the sample; and (c) comparing the level of expression or activity of the BMP-1 related protein to a standard level of expression or activity of the BMP-1 related protein. Methods of diagnosing the presence of squamous cell carcinoma in a subject are also provided, the methods comprising: (a) obtaining a sample; (b) detecting the level of expression of a BMP-1 related protein in the sample; and (c) comparing the level of expression or activity of the BMP-1 related protein in the sample to a standard level of expression or activity of the BMP-1 related protein, are also provided. Methods of diagnosing other cancers, including, e.g., glioma, are also provided.

In additional embodiments, the present invention provides methods of diagnosing the presence of a conditions characterized by increased expression or activity of laminin 5 in a subject, the methods comprising: (a) obtaining a sample; (b) detecting the level of expression or activity of laminin 5 or processed laminin 5 in the sample; and (c) comparing the level of expression or activity of laminin 5 or processed laminin 5 to a standard level of expression or activity of laminin 5 or processed laminin 5. Methods of diagnosing the presence of squamous cell carcinoma in a subject are also provided, the methods comprising: (a) obtaining a sample; (b) detecting the level of expression of laminin 5 or processed laminin 5 in the sample; and (c) comparing the level of expression or activity of laminin 5 or processed laminin 5 in the sample to a standard level of expression or activity of laminin 5 or processed laminin 5, are also provided.

In additional embodiments, the BMP-1 related protein is selected from the group consisting of BMP-1, mTld, mTll-1, and mTll-2. In further embodiments, the sample is a tissue sample, a urine sample, a serum sample, or a blood sample.

The invention also provides diagnostic kits for diagnosing the presence of a condition characterized by increased expression or activity of laminin 5, or for diagnosing the presence of a cancer, such as squamous cell carcinoma or glioma, in a sample from a subject, the kit comprising a means for detecting and measuring the level of BMP-1 related protein in the sample. The kit can comprise, e.g., an antibody reactive with, e.g., that binds to, a BMP-1 related protein, and a reagent that is capable of forming a complex with the BMP-1 related protein or with the antibody. In a preferred embodiment, the reagent is labeled. In further aspects, the antibody is specific for a protein selected from the group consisting of BMP-1, mTld, mTll-1, and mTll-2.

In another aspect, the present invention contemplates a method of screening for an agent that affects the processing of laminin 5 by BMP-1 related proteins is also provided, the method comprising: (a) contacting a sample containing unprocessed laminin 5 with at least one BMP related protein and the agent; (b) measuring the level of processed laminin 5 in the sample; (c) measuring the level of processed laminin 5 in a control sample; and (d) comparing the level of processed laminin 5 in the sample with the level of processed laminin 5 in a standard or control sample.

The present invention also relates to the identification of potential BMP-1 cleavage sequences within the α3 chain and the γ2 chain of laminin 5. A cleavage sequence within the γ2 chain of laminin 5 has been identified as CYSG/DENP (SEQ ID NO.: 1). Therefore, in one aspect, the present invention provides a polypeptide comprising a BMP-1 cleavage sequence, the polypeptide comprising the amino acid sequence of SEQ ID NO: 1. Cleavage sequences within the α3 chain of laminin 5 have been identified as LQFG/DIPT (SEQ ID NO.:2) (cleavage site is between glycine 1370 and aspartic acid 1371); QLLQ/DTPVA (SEQ ID NO.:3) (cleavage site is between glutamine 1337 and aspartic acid 1338) (Tsubota et al (2000) Biochem. Biophys Res. Commun. 278:614-620.); KVWQ/DACS (SEQ ID NO:4) (cleavage site is between glutamine and aspartic acid); and, QFAV/DMQT (SEQ ID NO:5) (cleavage site is between valine and aspartic acid). Accordingly, in further embodiments, the present invention provides isolated polypeptides comprising BMP-1 cleavage sequences, the polypeptides comprising an amino acid sequence selected from the group consisting of SEQ ID NO:2, SEQ ID NO:3, SEQ ID NO:4, and SEQ ID NO:5. Antibodies to these polypeptides are also provided. In various embodiments, the antibodies can be monoclonal, polyclonal, human, or humanized, and can be antibody fragments including Fab, F(ab′)₂, and Fᵥ fragments.

The present invention further contemplates methods of screening for agents that affect the processing of laminin 5 by BMP-1 related proteins. Methods are provided for identifying agents that inhibit the processing of laminin 5 by a BMP-1 related protein. In specific embodiments, the BMP-1 related protein is selected from the group consisting of BMP-1, mTld, mTll-1, and mTll-2. In one aspect, the method of screening for an agent that affects the processing of laminin 5 by BMP-1 related proteins comprises contacting a sample containing at least one polypeptide comprising SEQ ID NO:1 with a BMP-1 related protein and an agent; measuring the level of the polypeptide that is processed; measuring the level of the polypeptide that is processed in a control sample; and comparing the level of the polypeptide that is processed in the sample to the level of polypeptide that is processed in the control sample. In further embodiments, the at least one polypeptide can comprise SEQ ID NO:2, SEQ ID NO:3, SEQ ID NO:4, or SEQ ID NO:5.

In one embodiment of the present invention, expression of BMP-1 related proteins can be evaluated as a prognostic marker in conditions associated with increased expression or activity of laminin 5.

### BRIEF DESCRIPTION OF THE FIGURES

FIG. 1A, FIG. 1B, and FIG. 1C set forth the chemical structures of Compound 1, Compound 2, and Compound 3, respectively, three inhibitors of BMP-1 activity for use in the methods and assays of the present invention.

FIG. 2 sets forth results of processing of the laminin 5 α3 and γ2 chains by BMP-1 and MMP-2.

FIG. 3 sets forth data related to the effect of a BMP-1 related protein inhibitor on the processing of laminin 5 α3 and γ2 chains.

FIGS. 4A and 4B set forth data showing expression of BMP-1 in basal cells of the epidermis and basal follicular epithelium.

FIG. 5 sets forth data demonstrating dose-dependent inhibition of keratinocyte migration in the presence of a BMP-1 related protein inhibitor.

FIGS. 6A and 6B set forth data showing proteolytic processing of laminin 5 by BMP-1 related proteins, and the effects of inhibitors of BMP-1 related proteins on laminin 5 processing.

FIGS. 7A, 7B, 7C, and 7D set forth data relating to the effects of BMP-1 related protein inhibitors on migration of normal keratinocytes and of a squamous cell carcinoma cell line.

FIGS. 8A, 8B, 8C, and 8D set forth results showing dose-dependent inhibition of migration of normal immortalized keratinocytes by BMP-1 related protein inhibitors.

FIGS. 9A, 9B, 9C, and 9D set forth results showing dose-dependent inhibition of migration of squamous cell carcinoma cell line SCC-15 by BMP-1 related protein inhibitors.

FIGS. 10A, 10B, 10C, and 10D set forth results showing dose-dependent inhibition of migration of squamous cell carcinoma cell line SCC-25 by BMP-1 related protein inhibitors.

FIG. 11 sets forth results showing dose-dependent inhibition laminin 5 processing in normal immortalized keratinocytes by BMP-1 related protein inhibitors.

FIG. 12 sets forth results showing dose-dependent inhibition of laminin 5 processing in squamous cell carcinoma cell line SCC-15 by BMP-1 related protein inhibitors.

FIG. 13 sets forth results showing dose-dependent inhibition of laminin 5 processing in squamous cell carcinoma cell line SCC-25 by BMP-1 related protein inhibitors.

FIG. 14 sets forth data relating to the effects of BMP-1 related protein inhibitor on migration of keratinocyte cell line KLASV deficient in laminin 5 production.

FIGS. 15A, 15B, 15C, and 15D set forth data relating to the effects of BMP-1 related protein inhibitors on migration of KLASV cells plated on type I collagen substrate.

FIGS. 16A, 16B, 16C, and 16D set forth data relating to the effects of BMP-1 related protein inhibitor on migration of normal immortalized keratinocytes plated on type I collagen substrate.

FIGS. 17A, 17B, 17C, and 17D set forth data relating to the effects of BMP-1 related protein inhibitors on migration of squamous cell carcinoma cell line SCC-25 plated on a type I collagen substrate.

FIGS. 18A, 18B, 18C, and 18D set forth data relating to the effects of BMP-1 related protein inhibitors on migration of normal immortalized keratinocytes plated on plastic.

FIGS. 19A, 19B, 19C, and 19D set forth data relating to the effects of BMP-1 related protein inhibitors on migration of squamous cell carcinoma cell line SCC-25 plated on plastic.

FIG. 20 sets forth data relating to the effect of BMP-1 related protein inhibitors on the attachment of normal keratinocytes to processed and unprocessed laminin 5.

FIG. 21 sets forth data relating to the involvement of integrin α6β4 and integrin α3β1 in the attachment of normal keratinocytes to processed and unprocessed laminin 5.

FIG. 22 sets forth data relating to the effects of BMP-1 related protein inhibitors on invasion of squamous cell carcinoma cell line SCC-25.

FIG. 23 sets forth data relating to the effects of BMP-1 related protein inhibitors on invasion of squamous cell carcinoma cell line SiHa, which is unable to produce laminin 5.

FIG. 24 sets forth data relating to the effects of BMP-1 related protein inhibitors on invasion of squamous cell carcinoma cell line SCC-15.

FIG. 25 sets forth data relating to toxicity of BMP-1 related protein inhibitors on squamous carcinoma cell lines SCC-25, SCC-15, and SiHa.

FIGS. 26A and 26B set forth data showing co-localization of BMP-1 and laminin 5 in the invading buds of human skin squamous cell carcinoma.

### THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention. Each reference cited herein is incorporated herein by reference in its entirety.

The practice of the present invention will employ, unless otherwise indicated, conventional methods of chemistry, biochemistry, molecular biology, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

Definitions

The term “BMP-1 related proteins” as used herein refers to BMP-1 or to any related proteins or to any proteins that possess BMP-1 activity. Specifically, BMP-1 related proteins include BMP-1 and proteins arising from alternative mRNA splicing of the BMP-1 gene. These splice variants include, for example, mTld. The term “BMP-1 related proteins” additionally refers to separate gene products demonstrating BMP-1 activity, such as mTll-1 and mTll-2. Thus, the term “BMP-1 related proteins” specifically encompasses, but is not limited to, BMP-1, mTld, mTll-1, and mTll-2.

The term “BMP-1 activity” refers to the ability of a BMP-1 related protein to process a substrate protein, e.g., through cleavage of a specific cleavage site. In particular, the term “BMP-1 activity” as used herein refers to processing of laminin 5 by BMP-1 related proteins. Processing of laminin 5 can include cleavage of at least one of the laminin 5 chains, i.e., cleavage of the α3 chain, the β3 chain, or the γ2 chain of laminin 5.

A “BMP-1 inhibitor,” as that term is used herein, is an agent that inhibits or blocks BMP-1 activity. The term “BMP-1 inhibitor” contemplates agents that affect BMP-1 activity, specifically, that inhibit the activity of BMP-1 related proteins, such as, for example, BMP-1, mTld, mTll-1, and mTll-2. The agent can be chemical or synthetic or can be a naturally or recombinantly derived entity. Specifically, the term “BMP-1 inhibitor” refers to any agent that inhibits processing of laminin 5 by any BMP-1 related protein.

As used herein, the term “agent” includes any agent that affects the activity or expression of a target protein. An agent can be any peptide, polypeptide, antibody, nucleic acid, molecule, protein, carbohydrate, or any small molecule or other chemical agent that directly or indirectly affects the activity or expression of a target protein. Thus, an agent that affects the processing of laminin 5 by a BMP-1 related protein would be an agent that alters, such as by inhibition, the laminin 5 processing activity of a BMP-1 related protein.

The term “agonist” refers to a molecule which increases the extent or prolongs the duration of the effect of the biological activity of a BMP-1 related protein. Agonists may include proteins, nucleic acids, carbohydrates, small molecules, or any other agents, compounds, or molecules which bind to and/or modulate the effects of a BMP-1 related protein.

The term “antagonist” refers to a molecule which decreases the extent or duration of the effect of the biological activity of a BMP-1 related protein. Antagonists may include proteins, nucleic acids, carbohydrates, antibodies, small molecule inhibitors, or any other agents, compounds, or molecules which decrease the effects of a BMP-1 related protein.

The term “antibody” refers to intact molecules as well as to fragments thereof, such as Fab, F(ab′)₂, and Fv fragments, which are capable of binding an epitopic determinant. Antibodies that bind, or example, a BMP-1 related protein or laminin 5 can be prepared using intact polypeptides or using fragments containing small peptides of interest as the immunizing antigen. A polypeptide or oligopeptide used to immunize an animal (e.g., a mouse, rat, rabbit, etc.) can be derived from the translation of RNA, or synthesized chemically, and can be conjugated to a carrier protein if desired. Commonly used carriers chemically coupled to peptides are well known in the art and include, for example, bovine serum albumin, thyroglobulin, and keyhole limpet hemocyanin (KLH).

The term “antisense” refers to any composition containing a nucleic acid sequence which is complementary to the “sense” strand of a specific nucleic acid sequence. Antisense molecules may be produced by any method available in the art including by synthesis or transcription. Once introduced into a cell, the complementary nucleotides combine with natural sequences produced by the cell to form duplexes and to block either transcription or translation.

The terms “nucleic acid” or “polynucleotide” sequences or “polynucleotides” refer to oligonucleotides, nucleotides, or polynucleotides, or any fragments thereof, and to DNA or RNA of natural or synthetic origin which may be single- or double-stranded and may represent the sense or antisense strand, to peptide nucleic acid (PNA), or to any DNA-like or RNA-like material, natural or synthetic in origin. A polynucleotide fragment is any portion of a polynucleotide sequence that retains at least one structural or functional characteristic of the polynucleotide. Polynucleotide fragments can be of variable length, for example, greater than 60 nucleotides in length, at least 100 nucleotides in length, at least 1000 nucleotides in length, or at least 10,000 nucleotides in length.

“Altered” or “variant” polynucleotide sequences include those with deletions, insertions, or substitutions of different nucleotides resulting in a polynucleotide that encodes the same or a functionally equivalent polypeptide or polypeptides with selectively altered, i.e., enhanced or reduced, activity. Included within this definition are sequences displaying polymorphisms that may or may not be readily detectable using particular oligonucleotide probes or through deletion of improper or unexpected hybridization to alleles, with a locus other than the normal chromosomal locus for the subject polynucleotide sequence. A polynucleotide “variant” preferably has at least about 80%, more preferably at least about 90%, and most preferably at least about 95% polynucleotide sequence similarity to the particular polynucleotide sequence. It will be appreciated by those skilled in the art that as a result of the degeneracy of the genetic code, a multitude of variant polynucleotide sequences encoding a particular protein, some bearing minimal homology to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard codon triplet genetic code, and all such variations are to be considered as being specifically disclosed.

“Amino acid” or “polypeptide” sequence or “polypeptides” as these terms are used herein refer to an oligopeptide, peptide, or protein sequence, or to a fragment of any of these, and to naturally occurring or synthetic molecules. A polypeptide or amino acid “fragment” is any portion of a polypeptide sequence which retains at least one structural and/or functional characteristic of the polypeptide. Immunogenic fragments or antigenic fragments are fragments of BMP-1 related proteins, laminin 5, or BMP-1 related protein cleavage sequences within laminin 5, preferably, fragments of about five to fifteen amino acids in length, that retain at least one biological or immunological aspect of the protein. Where “amino acid sequence” is used to refer to the polypeptide sequence of a naturally occurring protein molecule, “amino acid sequence” and like terms are not meant to limit the amino acid sequence to the complete native sequence associated with the recited protein molecule.

“Altered” or “variant” polypeptides may contain deletions, insertions, or substitutions of amino acid residues which produce a silent change and result in a functionally equivalent polypeptide or may contain alterations that result in a polypeptide with selectively altered, i.e., enhanced or reduced, activity. Deliberate amino acid substitutions may be made on the basis of similarity in polarity, charge, solubility, hydrophobicity, hydrophilicity, and/or the amphipathic nature of the residues as long as the biological or immunological activity of the encoded polypeptide is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine. Analogous minor variations may also include amino acid deletions or insertions, or both. Preferably, amino acid variants retain certain structural or functional characteristics of a particular polypeptide. Guidance in determining which amino acid residues may be substituted, inserted, or deleted may be found, for example, using computer programs well known in the art, such as LASERGENE software (DNASTAR Inc., Madison, Wis.).

A molecule is said to be a “chemical derivative” of another molecule when it contains additional chemical moieties not normally a part of the molecule. Such moieties can improve the molecule's solubility, absorption, biological half-life, and the like. The moieties can alternatively decrease the toxicity of the molecule, eliminate or attenuate any undesirable side effect of the molecule, and the like. Moieties capable of mediating such effects are generally available in the art. (Gennaro, A. R., ed. (1990) Remington's Pharmaceutical Sciences, 18^{th} ed., Mack Publishing Co., Eaton, Pa.) Procedures for coupling such moieties to a molecule are well known in the art.

A “carrier” as the term is used herein is any inert substance used as a diluent, excipient, or vehicle in the formulation of a drug or other pharmaceutical composition, in order to confer a suitable consistency or form to the drug or pharmaceutical composition, and to aid in the application of the drug or pharmaceutical composition.

The term “functional equivalent” as it is used herein refers to a polypeptide or polynucleotide that possesses at least one functional and/or structural characteristic of a particular polypeptide or polynucleotide. A functional equivalent may contain modifications that enable the performance of a specific function. The term “functional equivalent” is intended to include fragments, mutants, hybrids, variants, analogs, or chemical derivatives of a molecule.

The terms “insertion” or “addition” refer to a change in a polypeptide or polynucleotide sequence resulting in the addition of one or more amino acid residues or nucleotides, respectively, as compared to the naturally occurring molecule.

The term “conditions associated with increased expression or activity of laminin 5” as it is used herein refers to conditions characterized by increased levels of processed laminin 5. The increased levels of processed laminin 5 could be due to increased processing of laminin 5, or could be due to increased expression of laminin 5, or both.

The phrase “BMP-1 related protein-associated disorders” as used herein refers to conditions and diseases associated with the expression or activity of BMP-1 related proteins. These include conditions associated with increased expression or activity of laminin 5, for example, cancers such as SCC and glioma. BMP-1 related protein-associated disorders include, but are not limited to, cancer, skin cancer, cancer of the head and neck, oral cancer, esophageal cancer, throat cancer, cervical cancer, lung cancer, colorectal cancer, bronchiogenic carcinoma, squamous cell carcinoma, glioma, and other forms of such disorders which have expanded locally by invasion or systemically by metastasis.

“Cancer” refers to any malignant tumor of potentially unlimited growth that expands locally by invasion and systemically by metastasis. Cancer also refers to any abnormal state marked by a cancer.

A “glioma” refers to any tumor or cancer originating in the neuroglia of the brain or spinal cord.

“Squamous cell carcinoma” refers to any malignant neoplasm or tumor of epithelial cells. Squamous cell carcinoma includes malignant tumors of epithelial origin that have expanded locally by invasion and/or systemically by metastasis. Epithelial cells include, for example, squamous cells, squamous carcinoma cells, keratinocytes, mucosal epithelial cells, e.g., oral mucosal cells, etc., gastrointestinal epithelial cells, corneal epithelium of the eye, and epithelial cells of the urinary and reproductive tract. SCC is commonly sun-induced, i.e., actinically derived SCC. SCC can also result from transplant or invasive surgery, or follow other immunosuppressive situations. Chronic inflammation can lead to development of SCC at the site of inflammation, e.g., a burn or other scar, Marjolin's ulcer, etc. SCC can be virally induced, for example, SCC can result from human papillomavirus-induced (HPV) infection. SCC can include adenoid (acantholytic) SCC, spindle cell SCC, verrucous carcinoma (VC), keratoacanthoma (KA), nodular SCC, periungual SCC, and other epithelial carcinomas.

The term “isolated” as used herein refers to a molecule separated not only from proteins, polynucleotides, etc., that are present in the natural source of the protein or polynucleotide, but also from other components in general, and preferably refers to a molecule found in the presence of, if anything, only a solvent, buffer, ion, or other component normally present in a solution of the same. As used herein, the terms “isolated” and “purified” do not encompass molecules present in their natural source.

The term “purified” as it is used herein denotes that the indicated molecule is present in the substantial absence of other biological macromolecules, e.g., polynucleotides, polypeptides, and the like. The term preferably contemplates that the molecule of interest is present in a solution or composition at least 80% by weight; preferably, at least 85% by weight; more preferably, at least 95% by weight; and, most preferably, at least 99.8% by weight. Water, buffers, and other small molecules, especially molecules having a molecular weight of less than about one kDa, can be present.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

The term “sample” is used herein in its broadest sense. Samples may be derived from any source, for example, from bodily fluids, secretions, tissues, cells, or cells in culture including, but not limited to, saliva, blood, urine, and organ tissue (e.g., biopsied tissue); from chromosomes, organelles, or other membranes isolated from a cell; from genomic DNA, cDNA, RNA, mRNA, etc.; and from cleared cells or tissues, or blots or imprints from such cells or tissues. A sample can be in solution or can be, for example, fixed or bound to a substrate. Methods for obtaining such samples are within the level of skill in the art.

### Detailed Description

The present invention relates to the discovery of the role of BMP-1 related proteins in processing laminin 5. The invention further relates to the ability of BMP-1 inhibitors to block processing of laminin 5 by BMP-1 related proteins. In particular, the invention related to the discovery that inhibition of laminin 5 processing by BMP-1 related proteins effectively attenuates the migration and invasion of cells, such as epithelial cells, including, for example, squamous cells, squamous carcinoma cells, keratinocytes, mucosal epithelial cells, gastrointestinal epithelial cells, corneal epithelium of the eye, and epithelial cells of the urinary and reproductive tract.

Therefore, in one embodiment, the present invention provides methods for treating a condition associated with increased expression or activity of laminin 5 by affecting the processing of laminin 5. In a specific embodiment, the invention contemplates methods of treating such conditions by affecting the processing of laminin 5 by BMP-1 related proteins. In a preferred embodiment, the invention provides methods of treating conditions associated with increased laminin 5 activity or expression by administering an agent that affects the processing of laminin 5 by BMP-1 related proteins.

Methods of treating squamous cell carcinoma are specifically provided herein. In one aspect, the present invention relates to methods of treating squamous cell carcinoma by affecting the processing of laminin 5 by BMP-1 related proteins. In specific embodiments, the squamous cell carcinoma is cancer of the urinary, reproductive, and gastrointestinal tract, lung cancer, pancreatic cancer, colorectal cancer, cutaneous cancer, oral cancer, lip cancer, tongue cancer, esophageal cancer, cervical cancer, or head and neck cancer. Methods for treating glioma are also contemplated herein.

The use of any agent that affects processing of laminin 5 by a BMP-1 related protein is specifically contemplated. The invention specifically provides for the use of any agent that inhibits the processing of laminin 5 by BMP-1 related proteins. Such an agent could include any one of a number of agents well known in the art. For example, the agent could be an antibody, a small molecule, an antisense sequence, a peptide, agonist, antagonist, etc. The agent could inhibit BMP-1 activity directly or indirectly, such as by binding to a BMP-1 related protein, and thus altering its activity, or by binding to laminin 5, and thus blocking the ability of a BMP-1 related protein to process laminin 5.

In one embodiment, the agent is a small molecule. In a further embodiment, the small molecule is a hydroxamate or hydroxamic acid or derivative thereof. See, e.g., International Publication No. WO 00/50390 and International Application No. PCT/US01/03410, incorporated by reference herein in their entireties.

The present invention further provides methods of screening for agents that affect the processing of laminin 5 by BMP-1 related proteins. In one aspect, the method encompasses identifying agents that inhibit the processing of laminin 5 by BMP-1 related proteins.

Squamous cell carcinoma (SCC), a malignant neoplasm of epithelial cells, is a common and dangerous form of cancer. SCC is highly invasive and associated with a high risk of metastasis, morbidity, and mortality. SCCs can arise in a wide range of tissues, and includes skin, lung, oral, cervical, gastric, and colorectal cancers, among others. The risk of SCC is strongly linked with environmental factors such as sun exposure and tobacco use. Thus, the skin, lips, mouth, throat, and esophagus are frequently affected. SCCs often invade neighboring tissues, such as, for example, the eye, and can also metastasize to other sites, such as, for example, to the lymph nodes, lung, and other distant sites. Surgical resection and radiotherapy are the most common forms of treatment. SCCs are associated with a high risk of recurrence, resulting in significant mortality. New therapeutic agents that specifically intervene in the local invasion and metastasis of SCC would be valuable additions to the current methods of treatment.

Laminin 5 is preferentially expressed by invading malignant cells of many human carcinomas in addition to squamous cell carcinomas, colon and mammary carcinomas (Pyke et al., Am. J. Pathol. 1994, 145(4):782-91) and in malignant gliomas (Fukushima et al., Int. J. Cancer 1998, 76: 63-72).

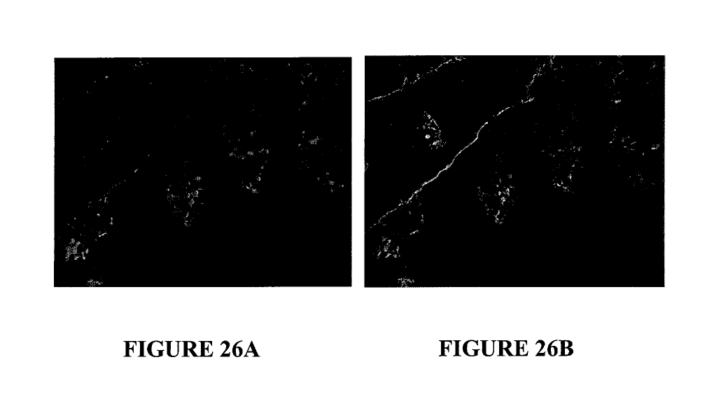

Immunofluorescent staining of human SCC tumor specimens reveals co-localization of BMP-1 and laminin 5 at the leading edge of invading tumor buds, showing the involvement of BMP-1 related proteins in laminin 5 processing and the stimulation of tumor invasion. (See FIGS. 26A and 26B.) Results demonstrated that, in the presence of BMP-1 inhibitors shown to inhibit BMP-1 related proteins including BMP-1, mTld, mTll-1, and mTll-2, the level of expression of laminin 5 remained unchanged, but the processing of both the α3 chain and the γ2 chain of laminin 5 was blocked. (See, e.g., Example 2 and Example 4.) In addition, particular BMP-1 inhibitors, designated herein as Compound 1, Compound 2, and Compound 3, were shown to have no toxic effect on cells and had optimal specificity for cleavage by BMP-1 related proteins. The compounds showed no inhibition of activity of MMP-1, MMP-2, MMP-9, or MT1-MMP, which are closely related to the BMP-1 class of proteins. These inhibitors were shown to potently inhibit the migration of keratinocytes and squamous carcinoma cells.

In one embodiment, present invention provides for methods of treating conditions associated with increased expression or activity of laminin 5, the method comprising administering an agent that affects the processing of laminin 5 by BMP-1 related proteins, wherein the agent is administered at the site of a surgical resection and additionally confers anti-scarring properties.

In another embodiment of the present invention, expression of BMP-1 related proteins is evaluated as a prognostic marker in tissues, e.g., in tumors, etc. In one embodiment, expression of BMP-1 related proteins is assessed as a prognostic marker in SCC tumors.

In another aspect, the present invention relates to methods of treating or preventing conditions associated with increased expression or activity of laminin 5 using an inhibitor of BMP-1 activity. In one embodiment, the inhibitor of BMP-1 activity is one of three highly specific and nontoxic BMP-1 inhibitors, designated herein as “Compound 1” and “Compound 2.” In another embodiment, the BMP-1 inhibitor is another highly specific agent with low cytotoxicity, designated herein as “Compound 3.”

The present invention also relates to the identification of cleavage sequences within the α3 chain and γ2 chain of laminin 5. BMP-1 γ2 chain and α3 chain cleavage sites were identified and confirmed by sequencing. A cleavage sequence within the γ2 chain was identified as CYSG/DENP (SEQ ID NO:1), with cleavage occurring between glycine 434 and aspartic acid 435. Cleavage sequences within the α3 chain were identified as LQFG/DIPT (SEQ ID NO:2), QLLQ/DTPVA (SEQ ID NO:3), KVWQ/DACS (SEQ ID NO:4), and QFAV/DMQT (SEQ ID NO:5), with cleavage occurring between glycine and aspartic acid, glutamine and aspartic acid, glutamine and aspartic acid, and valine and aspartic acid, respectively.

In one embodiment, the present invention provides isolated and purified polypeptides comprising SEQ ID NOs: 1, 2, 3, 4, and 5. Methods of using these polypeptides are also provided. For example, in one embodiment, the polypeptides can be used in methods of screening for agents that affect processing of laminin 5. The polypeptides could also be administered in vitro or in vivo to competitively affect processing of laminin 5. Further, these polypeptides could be used in assays directed to measurement of altered activity and expression of BMP-1 related proteins.

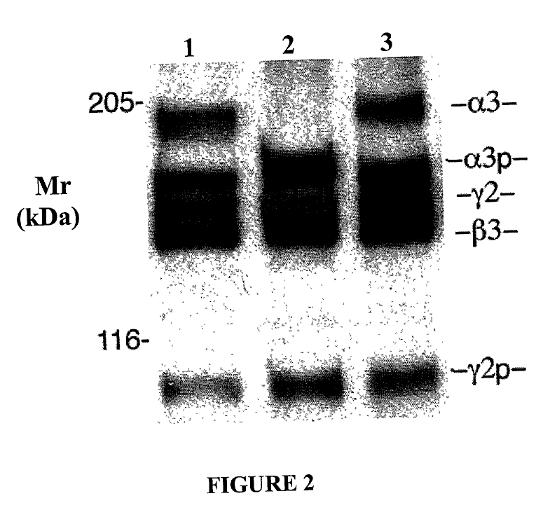

A series of metalloproteases were tested for the ability to process laminin 5. Results demonstrated that, for example, BMP-1, but not MMP-2, had activity on the laminin 5 substrate. Only the BMP-1 related proteins efficiently cleaved both the γ2 chain and α3 chain of human laminin 5 to sizes identical to endogenously processed laminin 5, as shown by immunoblot analysis. (See, e.g., FIG. 2.)

Although MMP-2 was enzymatically active (it readily processed collagen XVII and collagen IV), it failed to cleave human laminin 5, even at six times the enzyme-to-substrate ratio used for BMP-1 cleavage studies. Three highly selective inhibitors of BMP-1 related proteins showed significant potency in inhibiting BMP-1 activity, but not MMP-2 activity.

For example, Compound 2, an inhibitor of BMP-1 activity, and reactive with BMP-1 related proteins, inhibited laminin 5 α3 chain and γ2 chain processing and keratinocyte migration in a dose-dependent manner. This inhibition was shown to be non-toxic in cell culture and to have 500- to 1000-fold greater specificity for BMP-1 related proteins than for other MMPs, including MMP-2, as determined in enzyme-based assays. In the presence of 10 μM Compound 2, keratinocytes showed an almost complete lack of migration in transwells or over a scratch introduced across confluent cells to simulate a wound. A human squamous carcinoma cell line, SCC-25, showed an identical response to Compound 2.

BMP-1 inhibitors Compounds 1, Compound 2, and Compound 3 were also nontoxic to cultured cells even at 100 times inhibitory levels. Keratinocyte (KC) cultures containing 1 μM of either inhibitor showed a complete absence of laminin 5 processing by immunoblot. Each compound demonstrated inhibition of KC migration in a dose-dependent manner, as shown in Boyden chamber and scratch assays. Removal of the inhibitors restored normal cell migration. The inhibitors completely halted migration of KC and SCC cells, but did not inhibit the migration of KLASV cells, which are keratinocytes derived from the skin of patients with epidermolysis bullosa, and cultured for use in experiments. KLASV cells lack laminin 5, and the results suggest the inhibitors' effect on migration is specific to laminin 5 processing. Antibody blocking experiments showed increased binding of unprocessed laminin 5 with α3β1 integrin and processed laminin 5 with α6β4 integrin. These results demonstrate that BMP-1 related proteins process both the laminin 5 γ2 chain and α3 chain and that this processing is crucial in controlling cell migration, and that BMP-1 inhibitors can serve as highly specific and non-toxic anti-cancer agents.

Pharmaceutical Formulations and Routes of Administration

The present invention contemplates methods of treatment in which agents that affect the activity or expression of BMP-1 related proteins or of laminin 5 are administered, for example, in vivo, to affect the processing of laminin 5 by BMP-1 related proteins. These agents can be delivered directly or in pharmaceutical compositions along with suitable carriers or excipients, as well known in the art. Present methods of treatment include embodiments providing for administration of an effective amount of a compound or agent that inhibits the activity or expression of a BMP-1 related protein to a subject in need of treatment. Additionally, present methods of treatment can comprise administration of an effective amount of an agent that affects laminin 5 processing by BMP-1 related proteins, to a subject in need of such treatment. In a preferred embodiment, the subject is a mammalian subject, and in a most preferred embodiment, the subject is a human subject.

An effective amount of such sagents can readily be determined by routine experimentation, as can the most effective and convenient route of administration and the most appropriate formulation. Various formulations and drug delivery systems are available in the art. (See, e.g., Remington's Pharmaceutical Sciences, supra.)

Suitable routes of administration may, for example, include oral, rectal, transmucosal, transdermal, nasal, or intestinal administration and parenteral delivery, including intramuscular, subcutaneous, intramedullary injections, as well as intrathecal, direct intraventricular, intravenous, intraperitoneal, intranasal, or intraocular injections. The agent or composition thereof may be administered in a local rather than a systemic manner. For example, a suitable agent can be delivered via injection or in a targeted drug delivery system, such as a depot or sustained release formulation.

The pharmaceutical compositions of the present invention may be manufactured by any of the methods well-known in the art, such as by conventional mixing, dissolving, granulating, dragee-making, levigating, emulsifying, encapsulating, entrapping, or lyophilizing processes. As noted above, the compositions of the present invention can include one or more physiologically acceptable carriers such as excipients and auxiliaries that facilitate processing of active molecules into preparations for pharmaceutical use. Proper formulation is dependent upon the route of administration chosen.

For injection, for example, the composition may be formulated in aqueous solutions, preferably in physiologically compatible buffers such as Hanks's solution, Ringer's solution, or physiological saline buffer. For transmucosal or nasal administration, penetrants appropriate to the barrier to be permeated are used in the formulation. Such penetrants are generally known in the art. For oral administration, the agents can be formulated readily by combining the active agents with pharmaceutically acceptable carriers well known in the art. Such carriers enable the agents of the invention to be formulated as tablets, pills, dragees, capsules, liquids, gels, syrups, slurries, suspensions and the like, for oral ingestion by a subject. The agents may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agent doses.

Pharmaceutical preparations for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration.

For administration by inhalation, the agents for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges for use in an inhaler or insufflator may be formulated. These typically contain a powder mix of the agent and a suitable powder base such as lactose or starch.

Compositions formulated for parenteral administration by injection, e.g., by bolus injection or continuous infusion can be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Formulations for parenteral administration include aqueous solutions of the compound or agent to be administered, including in water-soluble form.

Suspensions of the active agents may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the agents to allow for the preparation of highly concentrated solutions. Alternatively, the active ingredient may be in powder form for constitution with a suitable vehicle, e.g., sterile pyrogen-free water, before use.

As mentioned above, the compositions of the present invention may also be formulated as a depot preparation. Such long acting formulations may be administered by implantation (for example, subcutaneously or intramuscularly) or by intramuscular injection. Thus, for example, the present agents may be formulated with suitable polymeric or hydrophobic materials (for example as an emulsion in an acceptable oil) or ion exchange resins, or as sparingly soluble derivatives, for example, as a sparingly soluble salt.

Suitable carriers for the hydrophobic molecules of the invention are well-known in the art and include co-solvent systems comprising, for example, benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system is effective in dissolving hydrophobic agents and produces low toxicity upon systemic administration. Naturally, the proportions of a co-solvent system may be varied considerably without destroying its solubility and toxicity characteristics. Furthermore, the identity of the co-solvent components may be varied. For example, other low-toxicity nonpolar surfactants may be used instead of polysorbate 80, the fraction size of polyethylene glycol may be varied, other biocompatible polymers may replace polyethylene glycol, e.g. polyvinyl pyrrolidone, and other sugars or polysaccharides may substitute for dextrose.

Alternatively, other delivery systems for hydrophobic molecules may be employed. Liposomes and emulsions are well known examples of delivery vehicles or carriers for hydrophobic drugs. Liposomal delivery systems are discussed above in the context of gene-delivery systems. Certain organic solvents such as dimethylsulfoxide also may be employed, although usually at the cost of greater toxicity. Additionally, the agents may be delivered using sustained-release systems, such as semi-permeable matrices of solid hydrophobic polymers containing the effective amount of the composition to be administered. Various sustained-release materials are established and available to those of skill in the art. Sustained-release capsules may, depending on their chemical nature, release the agents for a few weeks up to over 100 days. Depending on the chemical nature and the biological stability of the therapeutic reagent, additional strategies for protein stabilization may be employed.

For any composition used in the present methods of treatment, a therapeutically effective dose can be estimated initially using a variety of techniques well-known in the art. For example, in a cell culture assay, a dose can be formulated in animal models to achieve a circulating concentration range that includes the IC₅₀ as determined in cell culture. Where inhibition of BMP-1 activity is desired, for example, the concentration of the test agent that achieves a half-maximal inhibition of BMP-1 activity can be determined. Dosage ranges appropriate for human subjects can be determined, for example, using data obtained from cell culture assays and other animal studies.

A therapeutically effective dose of an agent refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD_{50/}ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

Dosage amount and interval may be adjusted individually to provide plasma levels or tissue levels of the active moiety which are sufficient to affect the expression or activity of BMP-1 related proteins, and/or of laminin 5, and/or or processing of laminin 5, as desired, i.e. minimal effective concentration (MEC). The MEC will vary for each agent but can be estimated from, for example, in vitro data, such as the concentration necessary to achieve 50-90% inhibition of laminin 5 processing using the assays described herein. Dosages necessary to achieve the MEC will depend on individual characteristics and route of administration. Agents or compositions thereof should be administered using a regimen which maintains plasma levels above the MEC for about 10-90% of the duration of treatment, preferably about 30-90% of the duration of treatment, and most preferably between 50-90%. In cases of local administration or selective uptake, the effective local concentration of the drug may not be related to plasma concentration.

The amount of agent or composition administered will, of course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a agent of the invention formulated in a compatible pharmaceutical carrier may also be prepared, placed in an appropriate container, and labeled for treatment of an indicated condition. Suitable conditions indicated on the label may include treatment of disorders or diseases, such as squamous cell carcinoma or glioma, or other cancers and conditions associated with altered expression or activity of laminin 5.

Antibodies

Antibodies directed to BMP-1 related proteins or to laminin 5 may be generated using methods well known in the art. Such antibodies may include, but are not limited to, polyclonal, monoclonal, chimeric, single chain antibodies, as well as Fab fragments, including F(ab′)₂ and Fᵥ fragments. Fragments can be produced, for example, by a Fab expression library. Neutralizing antibodies, i.e., those which inhibit BMP-1 activity, e.g., the processing of BMP-1 related proteins, or expression, or inhibit the expression or activity of laminin 5, are especially preferred for therapeutic use.

A target polypeptide, such as a BMP-1 related protein, laminin 5, or a specific polypeptide of a BMP-1 related protein cleavage sequence within laminin 5 or any laminin 5 chain (e.g., SEQ ID NOs:1 through 5), can be evaluated to determine regions of high immunogenicity. Methods of analysis and epitope selection are well-known in the art. (See, e.g., Ausubel et al., eds. (1988), Current Protocols in Molecular Biology.) Analysis and selection can also be accomplished, for example, by various software packages, such as LASERGENE NAVIGATOR software. (DNASTAR; Madison, Wis.) The polypeptides or fragments used to induce antibodies should be antigenic, but need not necessarily be biologically active. Preferably, an antigenic fragment or polypeptide is at least 5 amino acids in length, more preferably, at least 10 amino acids in length, and most preferably, at least 15 amino acids in length. It is preferable that the antibody-inducing fragment or polypeptide is identical to at least a portion of the amino acid sequence of the target polypeptide, e.g., a BMP-1 related protein, laminin 5, or a specific BMP-1 related protein cleavage sequence within laminin 5, or fragments or subunits thereof. A peptide or fragment that mimics at least a portion of the sequence of the naturally occurring target polypeptide can also be fused with another protein, e.g., keyhole limpet hemocyanin (KLH), and antibodies can be produced against the chimeric molecule.

Methods for the production of antibodies are well-known in the art. For example, various hosts, including goats, rabbits, rats, mice, humans, and others, may be immunized by injection with the target polypeptide or any immunogenic fragment or peptide thereof. Depending on the host species, various adjuvants may be used to increase immunological response. Such adjuvants include, but are not limited to, Freund's adjuvant, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Monoclonal and polycolonal antibodies may be prepared using any technique which provides for the production of antibody molecules by continuous cell lines in culture. Techniques for in vivo and in vitro production are well-known in the art. (See, e.g., Pound, J. D. (1998) Immunochemical Protocols, Humana Press, Totowa N.J.; Harlow, E. and D. Lane (1988) Antibodies, A Laboratory Manual, Cold Spring Harbor Laboratory, New York.) The production of chimeric antibodies is also well-known, as is the production of single-chain antibodies. (See, e.g., Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454.) Antibodies with related specificity, but of distinct idiotypic composition, may be generated, for example, by chain shuffling from random combinatorial immunoglobin libraries. (See, e.g., Burton D. R., (1991) Proc. Natl. Acad. Sci. 88:11120-11123.)

Antibodies may also be produced by inducing in vivo production in the lymphocyte population or by screening immunoglobulin libraries or panels of highly specific binding reagents. (See, e.g., Orlandi, R. et al. (1989) Proc. Natl. Acad. Sci. 86:3833-3837; Winter, G. and C. Milstein (1991) Nature 349:293-299.) Antibody fragments which contain specific binding sites for the target polypeptide may also be generated. Such antibody fragments include, but are not limited to, F(ab′)₂ fragments, which can be produced by pepsin digestion of the antibody molecule, and Fab fragments, which can be generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 254:1275-1281.)

Antibodies can be tested for anti-target polypeptide activity using a variety of methods well-known in the art. Various techniques may be used for screening to identify antibodies having the desired specificity, including various immunoassays, such as enzyme-linked immunosorbent assays (ELISAs), including direct and ligand-capture ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art. (See, e.g., Harlow and Lane, supra.) Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed. (See, e.g. Maddox, D. E., et al (1983) J Exp Med 158:1211.)

Antibodies as described above could also be used to identify BMP-1 related proteins, laminin 5 or processed laminin 5 or laminin 5 chains, or fragments or subunits thereof, in a sample, e.g., from biopsied tissue, etc. The amount of BMP-1 related protein or laminin 5 present could be determined, for example, by quantitative image analysis. BMP-1 related protein or laminin 5 mRNA levels could also be determined, e.g., by reverse transcriptase polymerase chain reaction (RT-PCR) using portions of the biopsied tissue or by other methods well known in the art. In particular, in this method, mRNA from a tissue sample, in total, or that specific for a BMP-1 related protein, laminin 5, laminin 5 chains, or fragments or subunits thereof, could be transcribed to DNA and then amplified through PCR using specific primer sequences. Quantitation of mRNA corresponding to BMP-1 related proteins, for example, could be determined, for example, by a competition reaction using equal volumes of the patient sample run against a series of decreasing known concentrations, e.g., of a mimic or mutant cDNA fragment.

The present invention contemplates the use of antibodies specifically reactive with a BMP-1 related protein, laminin 5, laminin 5 chains, or a specific BMP-1 related protein cleavage sequence within laminin 5 or any of the laminin 5 chains, or fragments or subunits thereof, that neutralize the biological activity of a BMP-1 related protein or laminin 5, thus inhibiting processing of laminin 5 by BMP-1 related proteins. The antibody administered in the method can be the intact antibody or antigen binding fragments thereof, such as Fab, F(ab′)₂, and Fᵥ fragments, which are capable of binding the epitopic determinant. The antibodies used in the method can be polyclonal or, more preferably, monoclonal antibodies. Monoclonal antibodies with different epitopic specificities are made from antigen-containing fragments of the protein by methods well known in the art. (See Ausubel et al., supra.)

In the present invention, therapeutic applications include those using “human” or “humanized” antibodies directed to a BMP-1 related protein, laminin 5, laminin 5 chains, or a specific BMP-1 related protein cleavage sequence within laminin 5 or any of the laminin 5 chains, or fragments or subunits thereof. Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a BMP-1 related protein, laminin 5, laminin 5 chain, or a specific BMP-1 related protein cleavage sequence within laminin 5 or any of the laminin 5 chains, is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

Antisense

The present invention provides for a therapeutic approach which affects expression and activity of BMP-1 related proteins or laminin 5 by inhibiting the expression of BMP-1 related proteins or laminin 5. Specifically, a therapeutic approach which directly interrupts the translation of BMP-1 related proteins, laminin 5, or any chain of laminin 5 mRNA could be used to alter the expression and activity of BMP-1 related proteins, laminin 5, or any chain of laminin 5.

Antisense technology relies on the modulation of expression of a target protein through the specific binding of an antisense sequence to a target sequence encoding the target protein or directing its expression. (See, e.g., Agrawal, S., ed. (1996) Antisense Therapeutics, Humana Press Inc., Totawa N.J.; Alama, A. et al. (1997) Pharmacol Res. 36(3):171-178; Crooke, S. T. (1997) Adv. Pharmacol. 40:1-49; and Lavrosky, Y. et al. (1997) Biochem. Mol. Med. 62(1):11-22.) Antisense sequences are nucleic acid sequences capable of specifically hybridizing to at least a portion of a target sequence. Antisense sequences can bind to cellular mRNA or genomic DNA, blocking translation or transcription and thus interfering with expression of a targeted protein product. Antisense sequences can be any nucleic acid material, including DNA, RNA, or any nucleic acid mimics or analogs. (See, e.g., Rossi, J. J. et al. (1991) Antisense Res. Dev. 1(3):285-288; Pardridge, W. M. et al. (1995) Proc. Nat. Acad. Sci. 92 (12):5592-5596; Nielsen, P. E. and G. Haaima (1997) Chem. Soc. Rev. 96:73-78; and Lee, R. et al. (1998) Biochemistry 37 (3):900-1010.) Delivery of antisense sequences can be accomplished in a variety of ways, such as through intracellular delivery using an expression vector. Site-specific delivery of exogenous genes is also contemplated, such as techniques in which cells are first transfected in culture and stable transfectants are subsequently delivered to the target site.

Antisense oligonucleotides of about 15 to 25 nucleic acid bases are typically preferred as such are easily synthesized and are capable of producing the desired inhibitory effect. Molecular analogs of antisense oligonucleotide may also be used for this purpose and can have added advantages such as stability, distribution, or limited toxicity advantageous in a pharmaceutical product. In addition, chemically reactive groups, such as iron-linked ethylenediamine-tetraacetic acid (EDTA-Fe), can be attached to antisense oligonucleotides, causing cleavage of the RNA at the site of hybridization. These and other uses of antisense methods to inhibit the in vitro translation of genes are well known in the art. (See, e.g., Marcus-Sakura (1988) Anal. Biochem 172:289.)

Delivery of antisense therapies and the like can be achieved intracellularly through using a recombinant expression vector such as a chimeric virus or a colloidal dispersion system which, upon transcription, produces a sequence complementary to at least a portion of the cellular sequence encoding the target protein. (See, e.g., Slater, J. E. et al. (1998) J. Allergy Cli. Immunol. 102 (3):469-475.) Delivery of antisense sequences can also be achieved through various viral vectors, including retrovirus and adeno-associated virus vectors. (See, e.g., Miller, A. D. (1990) Blood 76:271; and Uckert, W. and W. Walther (1994) Pharacol. Ther. 63(3):323-347.) Vectors which can be utilized for antisense gene therapy as taught herein include, but are not limited to, adenoviruses, herpes viruses, vaccinia, or, preferably, RNA viruses such as retroviruses.

Retroviral vectors are preferably derivatives of murine or avian retrovirus. Retroviral vectors can be made target-specific by inserting, for example, a polynucleotide encoding a protein or proteins such that the desired ligand is expressed on the surface of the viral vector. Such ligand may be a glycolipid carbohydrate or protein in nature. Preferred targeting may also be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the antisense polynucleotide.

Recombinant retroviruses are typically replication defective, and can require assistance in order to produce infectious vector particles. This assistance can be provided by, for example, using helper cell lines that contain plasmids encoding all-of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal may be used. These cell lines produce empty virions, since no genome is packaged. If a retroviral vector is introduced into such cells in which the packaging signal is intact, but the structural genes are replaced by other genes of interest, the vector can be packaged and vector virion produced.

Other gene delivery mechanisms that can be used for delivery of antisense sequences to target cells include colloidal dispersion and liposome-derived systems, artificial viral envelopes, and other systems available to one of skill in the art. (See, e.g., Rossi, J. J. (1995) Br. Med. Bull. 51 (1):217-225; Morris, M. C. et al. (1997) Nucl. Acids Res. 25 (14):2730-2736; and Boado, R. J. et al. (1998) J. Pharm. Sci. 87 (11):1308-1315.) For example, delivery systems can make use of macromolecule complexes, nanocapsules, microspheres, beads, and lipid-based systems including oil-in-water emulsions, micelles, mixed micelles, and liposomes.

In one embodiment, the present invention provides a colloidal delivery system that uses liposomes. Liposomes are artificial membrane vesicles useful as delivery vehicles in vitro and in vivo. It has been shown that large unilamellar vesicles (LUV), ranging in size from about 0.2 to about 4.0 μm can encapsulate a substantial percentage of an aqueous buffer containing large macromolecules. RNA, DNA, and intact virions, for example, can be encapsulated within the aqueous interior and delivered to cells in a biologically active form. (See, e.g., Fraley, et al. (1981) Trends Biochem. Sci., 6:77.)

Liposomes have been used for delivery of polynucleotides in, for example, mammalian, plant, yeast, and bacterial cells. In order for a liposome to be an efficient gene transfer vehicle, the following characteristics should be present: (1) highly efficient encapsulation of the genes of interest, without comprising the biological activity of these genes; (2) preferential and substantial binding to target cells in comparison to non-target cells; (3) highly efficient delivery of vesicle contents to target cell cytoplasm; and (4) accurate and effective expression of genetic information. (See, e.g., Mannino et al. (1988) Biotechniques 6:682.) The composition of the liposome is usually a combination of phospholipids, particularly high-phase-transition-temperature phospholipids, usually in combination with steroids, especially cholesterol. Other phospholipids or other lipids may also be used.

Physical characteristics of liposomes depend on pH, ionic strength, and the presence of divalent cations. Examples of lipids useful in liposome production include phosphatidyl agents, such as phosphatidylglycerol, phosphatidylcholine, phosphatidylserine, phosphatidylethanolamine, sphingolipids, cerebrosides, and gangliosides. Particularly useful are diacylphosphatidylglycerols, in which the saturated lipid moiety contains from 14 to 18 carbon atoms, particularly from 16 to 18 carbon atoms. Illustrative phospholipids include egg phosphatidylcholine, dipalmitoylphosphatidycholine and distearoylphosphatidylcholine.

The targeting of liposomes has been classified based on anatomical and mechanistic factors. Anatomical classification is based on the level of selectivity, for example, organ-specific, cell-specific, and organelle-specific liposomes. Mechanistic targeting can be distinguished based upon whether it is passive or active. Passive targeting utilizes the natural tendency of liposomes to distribute to cells of the reticulo-endothelial system (RES) in organs containing sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cells types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. In general, the agents bind to the surface of the targeted delivery system to find and interact with the desired cells. A ligand may be any agent of interest which will bind to another agent, such as a receptor.

Diagnostics

Another aspect of the present invention provides methods for diagnosing cancer, squamous cell carcinoma, glioma, or any condition associated with altered expression or activity of a BMP-1 related protein, or laminin 5, or processing of laminin 5. In one embodiment of the present invention, the level of a BMP-1 related protein in a biological sample can be detected. A comparison of the amount of a BMP-1 related protein in the sample to that in a non-disease sample can provide an indication of whether BMP-1 related protein expression and activity is altered, and thus indicate whether the subject has or is at risk for a disorder such as cancer. In another embodiment of the present invention, the level of laminin 5, processed laminin 5, or processed chains of laminin 5, relating to the expression and activity of laminin 5, in a biological sample can be detected. A comparison of the amount laminin 5, processed laminin 5, or processed chains of laminin 5 in the sample to that in a non-disease sample can provide an indication of whether BMP-1 related protein expression and activity or processing of laminin 5 is altered, and thus indicate whether the subject has or is at risk for a disorder, such as cancers associated with increased expression or activity of laminin 5, e.g., SCC, glioma, etc. The diagnostics tools and methods of the present invention may also include imaging systems known in the art. Further, BMP-1 related proteins or fragments or subunits thereof, antibodies to BMP-1 related proteins, and compounds or agents that effect expression or activity of BMP-1 related proteins can also be included in a kit for detection of disorders associated with altered expression and activity of BMP-1 related proteins. For example, diagnostic kits for assays utilizing radioimmunoassay (RIA), fluorescent immunoassay, or ELISA (enzyme-linked immunoabsorbent assay) techniques are specifically contemplated.

The present invention is further directed to a method of detecting or diagnosing the presence of a pathology characterized by altered expression or activity of laminin 5 in a sample from a subject, in particular, those associated with decreased or increased expression and activity of processed laminin 5 or any processed chain of laminin 5.

In a preferred method, the detection or diagnosis is accomplished by measuring levels of BMP-1 related protein, laminin 5, or processed laminin 5 in a sample from a subject. In one aspect, the subject is a human subject. In one embodiment, the method includes determining the level of BMP-1 related protein, laminin 5, or processed laminin 5 in a tissue biopsy sample and comparing this level to the level of BMP-1 related protein, laminin 5, or processed laminin 5 present in a tissue biopsy from a normal tissue or sample, i.e., a sample from a subject without a BMP-1 related protein- or laminin 5-associated disorder, such as cancer, squamous cell carcinoma, and glioma. An elevated level of BMP-1 related protein expression or activity or an elevated level of laminin 5 expression or activity in the first sample is indicative of the pathological condition in question.

More generally, detection of BMP-1 related proteins, laminin 5, or processed laminin 5, may be obtained through immunoassay methods, for example, using ELISAs, RIAs, or any other assays which utilize an antibody to detect the presence of a protein marker. The ELISA and RIA methods are preferred and may be used, for example, with the polyclonal or monoclonal antibodies of the present invention to detect levels of BMP-1 related proteins, laminin 5, or processed laminin 5. Levels of BMP-1 related proteins, laminin 5, or processed laminin 5 in this first sample are measured, for example, through immunoassay, and are compared with the BMP-1 related proteins, laminin 5, or processed laminin 5 levels in a second sample, the second sample being obtained from a patient known to have a BMP-1 related protein-associated disorder or from a patient known not to have any BMP-1 related protein-associated disorder, to determine the presence or progression of such a disorder. The same methods may be used to monitor the progression of a BMP-1 related protein-associated disorder.

More generally, antibodies specific for a target polypeptide, such as antibodies specific for a BMP-1 related protein, for laminin 5, laminin 5 chains, or for specific BMP-1 related protein cleavage sequences within laminin 5 or chains of laminin 5, or fragments or subunits thereof, are useful in the present invention for diagnosis of BMP-1 related protein-associated disorders. The present diagnostic assays include methods utilizing the antibody and a label to detect a BMP-1 related protein, laminin 5, laminin 5 chains, or specific BMP-1 related protein cleavage sequences within laminin 5 or chains of laminin 5, or fragments or subunits thereof, in a sample from a patient suspected of having a BMP-1 related protein-associated disorder. The sample could comprise, for example, body fluids, cells, tissues, or extracts of such tissues, including, for example, cells micro-dissected from biopsy material. Protocols employed to screen for and identify antibodies having the desired specificity can also be used for the detection of a BMP-1 related protein, laminin 5, laminin 5 chains, or specific BMP-1 related protein cleavage sequences within laminin 5 or chains of laminin 5, or fragments or subunits thereof or fragments or subunits thereof in the sample.

Preferably, in the diagnostic methods of the present invention, normal or standard values for BMP-1 related protein expression, laminin 5 expression or activity, or processed laminin 5 expression or activity are established in order to provide a basis for the diagnosis of the existence of a BMP-1 related protein-associated disorder or a predisposition to a BMP-1 related protein-associated disorder. In one of the methods of the present invention, this is accomplished by combining body fluids, tissue biopsies, or cell extracts taken from normal subjects with antibody to a BMP-1 related protein, laminin 5, laminin 5 chain, or processed laminin 5 under conditions suitable for complex formation. Such conditions are well known in the art. The amount of standard complex formation may be quantified by comparing levels of antibody-target complex in the normal sample with a dilution series of positive controls, in which a known amount of antibody is combined with known concentrations of purified BMP-1 related protein, laminin 5, laminin 5 chains, or processed laminin 5, or fragments or subunits thereof. Standard values obtained from normal samples may be compared, for example, in a specific embodiment, with values obtained from samples from subjects suspected of having a BMP-1 related protein-associated disorder, or having a predisposition to a BMP-1 related protein-associated disorder. Deviation between standard and subject values establishes the presence of or predisposition to the disease state. The diagnostic methods of the present invention may also be directed to the detection of a predisposition or susceptibility to a cancer, such as, for example, squamous cell carcinoma or glioma.

Monoclonal or polyclonal antibodies can be detected by methods discussed, for example, infra. Monoclonal or polyclonal antibodies against a BMP-1 related protein, laminin 5, laminin 5 chains, processed laminin 5, or to peptides corresponding to BMP-1 related protein cleavage sequences within laminin 5 can be conjugated to an appropriate enzyme such as horseradish peroxidase, protein ferritin, enzyme alkaline phosphatase, β-D-galactosidase, etc. These enzyme-linked antibody preparations can be mixed with, for example, biopsy samples that contain unknown amounts of BMP-1 related protein, laminin 5, laminin 5 chains, processed laminin 5, or to peptides corresponding to BMP-1 related protein cleavage sequences within laminin 5, bound or unbound, in an indirect ELISA. Direct or sandwich ELISAs could also be performed using the same antibodies.

RIA techniques may also be used to measure levels of the BMP-1 related protein, laminin 5, laminin 5 chains, or processed laminin 5 in, for example, a tissue biopsy. For example, BMP-1 related protein, laminin 5, laminin 5 chains, processed laminin 5, or peptides corresponding to BMP-1 related protein cleavage sequences within laminin 5, or fragments or subunits thereof, may be radioactively labeled and mixed with monoclonal or polyclonal antibodies specific for a BMP-1 related protein, laminin 5, laminin 5 chains, processed laminin 5, or peptides corresponding to BMP-1 related protein cleavage sequences within laminin 5, and a serum or tissue sample containing an unknown amount of unlabeled BMP-1 related protein, laminin 5, laminin 5 chains, or processed laminin 5. The labeled and unlabeled BMP-1 related protein, laminin 5, laminin 5 chains, processed laminin 5, or peptides corresponding to BMP-1 related protein cleavage sequences within laminin 5 compete for binding with the monoclonal or polyclonal antibody. By measuring the amount of radioactivity of the reaction mixture, the amount of BMP-1 related protein, laminin 5, laminin 5 chains, or processed laminin 5 present in the sample can be quantitatively determined. See, e.g., U.S. Pat. Nos. 4,438,209 and 4,591,573. Non-competitive RIAs can also be performed.

Polynucleotide sequences encoding a BMP-1 related protein or laminin 5, or fragments or subunits thereof, can be used for the diagnosis of conditions or diseases associated with increased levels of BMP-1 related protein expression and activity or associated with increased levels of laminin 5 expression or activity. For example, polynucleotide sequences encoding a BMP-1 related protein may be used in hybridization or PCR assays of fluids or tissues from biopsies to detect BMP-1 related protein expression and activity. The form of such qualitative or quantitative methods may include Southern or northern analysis, dot blot or other membrane-based technologies; PCR technologies; dip stick, pin, chip and ELISA technologies. All of these techniques are well known in the art and are the basis of many commercially available diagnostic kits.

The present invention provides kits for detecting a BMP-1 related protein, laminin 5, laminin 5 chains, or processed laminin 5 in samples, in particular, in fluid samples or tissue biopsy samples. In a particular embodiment, this kit comprises a monoclonal and/or polyclonal antibody specific for a BMP-1 related protein, or fragments or subunits thereof, bound to a support and a second monoclonal and/or polyclonal antibody specific for a different BMP-1 related protein epitope and enzyme-labeled. The kit further comprises reagents for detecting the enzyme-labeled monoclonal and/or polyclonal antibody. The reagent kit employs immunological methods in measuring BMP-1 related protein in the tissue sample, thus allowing for the detection and monitoring of cancer, in particular, squamous cell carcinoma. In another embodiment, the kit comprises a radio-labeled or fluorescein labeled antibody in place of the enzyme-labeled antibody.

In one embodiment, the diagnostic kit of the present invention comprises elements useful in the detection of a BMP-1 related protein, laminin 5, chains of laminin 5, or processed laminin 5 in tissue samples, using immuno-histochemical techniques. The kit could be used in conjunction with, for example, a software program which allows for quantitative measurement of the levels of a BMP-1 related protein, laminin 5, chains of laminin 5, or processed laminin 5 in the tissue sample by image analysis or other comparative techniques. Another embodiment provides a diagnostic kit for detecting and measuring levels of BMP-1 related protein mRNA in tissue samples. In one embodiment, the kit comprises reagents used to reverse transcribe BMP-1 related protein mRNA to DNA. The kit can further comprise reagents necessary to amplify BMP-1 related protein-specific DNA, including primers complementary to polynucleotides encoding BMP-1 related protein, or fragments or subunits thereof. The kit can also include a competitive mimic or mutant cDNA for use in quantifying the level of BMP-1 related protein mRNA present in the sample.

In a preferred embodiment, the diagnostic kit of the present invention is packaged and labeled, for example, in box or container which includes the necessary elements of the kit, and includes directions and instructions on the use of the diagnostic kit, as well as any ancillary reagents.

Methods for Screening

The present invention additionally contemplates methods for screening for agents that affect the processing of laminin 5. The methods of the claimed invention also include the use of the BMP-1 related proteins, laminin 5, chains of laminin 5, processed laminin 5, polypeptides corresponding to BMP-1 related protein cleavage sequences within laminin 5 (e.g., SEQ ID NOs: 1 through 5), or fragments or subunits thereof, to screen for or otherwise identify agents, including agonists or antagonists, which directly or indirectly affect a BMP-1 related protein or laminin 5 of the present invention. Agents that bind to a BMP-1 related protein, to laminin 5, or to a chain of laminin 5, may inhibit the activities of these proteins. The agents can include, for example, antibodies and fragments thereof, small molecules, polypeptides (synthetic, natural, or enzymatically- or recombinantly-produced), and aptamers.

The screening methods of the present invention can directly test for the binding of an agent to a BMP-1 related protein or to laminin 5. Alternatively, screening assays can test for binding of a candidate agent in the presence of a labeled competitor. Binding can be detected by a number of methods available in the art, including, for example, fluorophores, enzyme conjugates, radioisotopes, or any detectable label.

In one aspect, assays of the present invention include contacting a BMP-1 related protein or fragments or subunits thereof with the candidate agent, detecting a level of BMP-1 related protein activity or expression, and comparing that level of activity or expression to a standard level obtained by methods known in the art. These methods could involve, for example, BMP-1 related proteins affixed to solid supports, cell-free preparations, or natural or synthetic product mixtures. Assays, such as ELISAs, can be designed in which antibodies, monoclonal or polyclonal, bind directly or indirectly to a BMP-1 related protein or compete with BMP-1 related protein for binding.

The screening methods of the present invention can be used to identify agents that can be used in methods for treating the previously described BMP-1 related protein-associated diseases, disorders, and conditions. Agents identified using the present methods can be administered to produce the desired effect, such inhibiting BMP-1 related protein activity, such as the processing and cleavage of laminin 5 in a subject. Additionally, the present invention provides methods for the identification of agents which may decrease BMP-1 related protein activity in specific cells or tissues as desired under certain conditions.

In order to identify agents for use in treating or preventing a BMP-1 related protein-associated disorder by modulating the activity and expression of a BMP-1 related protein, BMP-1 related proteins, laminin 5, laminin 5 chains, or fragments or subunits thereof, can be used for screening therapeutic agents in any of a variety of screening techniques. Fragments employed in such screening tests may be free in solution, affixed to a solid support, borne on a cell surface, or located intracellularly. The blocking or reduction of biological activity or the formation of binding complexes between a BMP-1 related protein or laminin 5 and the agent being tested can be measured by methods available in the art.

Other techniques for drug screening which provide for a high throughput screening of agents having suitable binding affinity to a BMP-1 related protein or to laminin 5, or to another target polypeptide useful in modulating, regulating, or inhibiting the expression and/or activity of a BMP-1 related protein or laminin 5, are known in the art. For example, microarrays carrying test agents can be prepared, used, and analyzed using methods available in the art. (See, e.g., Shalon, D. et al. (1995) PCT Application No. WO 95/35505; Baldeschweiler et al. (1995) PCT Application No. WO95/251 116; Brennan, T. M. et al. (1995) U.S. Pat. No. 5,474,796; Heller, M. J. et al, (1997) U.S. Pat. No. 5,605,662.)

Identifying small agents that modulate BMP-1 related protein activity can also be conducted by various other screening techniques, which can serve to identify antibodies and other agents that interact with a BMP-1 related protein or laminin 5 and can be used as drugs and therapeutics in the present methods. (See, e.g., Enna, S. J. et al., eds. (1998) Current Protocols in Pharmacology, John Wiley and Sons.) Assays will typically provide for detectable signals associated with the binding of the agent to a protein or cellular target. Binding can be detected by, for example, fluorophpres, enzyme conjugates, and other detectable labels well-known in the art. (Id.) The results may be qualitative or quantitative.

For screening the agents for specific binding, various immunoassays may be employed for detecting, for example, human or primate antibodies bound to the cells. Thus, one may use labeled anti-hlg, e.g., anti-hlgM, hlgG, or combinations thereof to detect specifically bound human antibody of the galactosyl epitope. Various labels can be used such as radioisotopes, enzymes, fluorescers, chemiluminescers, particles, etc. There are numerous commercially available kits providing labeled anti-hlg, which may be employed in accordance with the manufacturer's protocol.

For screening the agents for cytotoxic effects, a wide variety of protocols may be employed to ensure that one has the desired activity. One will normally use cells, which may be naturally occurring or modified, cell lines, or the like. The cells may be prokaryotic or eukaryotic. For example, if one is interested in a pathogen, where it does not matter to which epitope the agent conjugate binds, one can combine the pathogenic cells with each of the agents in the presence of an antibody dependent cytotoxic system to determine the cytotoxic effect. One may perform this assay either prior to or subsequent to determining the effect of the various candidate agents on cells of the host to whom the agent would be administered. In this way, one would obtain a differential analysis between the affinity for the pathogenic target and the affinity for host cells which might be encountered, based on the mode of administration.

In some situations, one would be interested in a particular cellular status, such as an activated state, as may be present with T cells in autoimmune diseases, transplantation, and the like. In this situation one would first screen the agents to determine those which bind to the quiescent cell, and as to those agents which are not binding to the quiescent cells, and screen the remaining candidate agents for cytotoxicity to the activated cells. One may then screen for other cells present in the host which might be encountered by the agents to determine their cytotoxic effect. Alternatively, one might employ cancer cells and normal cells to determine whether any of the agents have higher affinity for the cancer cells, as compared to the normal cells. Again, one could screen the library of agents for binding to normal cells and determine the effect.

Those agents which are not cytotoxic to normal cells could then be screened for their cytotoxic effect to cancer cells. Even where some cytotoxicity exists for normal cells, in the case of cancer cells, where there is a sufficient differentiation in cytotoxic activity, one might be willing to tolerate the lower cytotoxicity for normal cells, where the agent is otherwise shown to be effective with cancer cells.

Instead of using cells which are obtained naturally, one may use cells which have been modified by recombinant techniques. Thus, one may employ cells which can be grown in culture, which can be modified by upregulating or downregulating a particular gene. In this way, one would have cells that differ as to a single protein on the surface. One could then differentially assay the library as to the effect of members of the library on cells for which the particular protein is present or absent. In this way, one could determine whether the agent has specific affinity for a particular surface membrane protein as distinct from any of the proteins present on the surface membrane.

One may differentiate between cells by using antibodies binding to a particular surface membrane protein, where the antibodies do not initiate the complement dependent cytotoxic effect, for example, using different species, isotypes, or combinations thereof. By adding the antibodies, blocking antisera or monoclonal antibodies, to one portion of the cells, those cells will not have the target protein available for binding to the library member. In this way one creates comparative cells which differ in their response based on the unavailability in one group of a single protein. While antibodies will usually be the most convenient reagent to use, other specific binding entities may be employed which provide the same function.

For use in the assay to determine binding, one may use an antibody-dependent cytotoxic system. One could use synthetic mixtures of the ingredients, where only those components necessary for the cytotoxic effect are present. This may be desirable where components of blood or plasma may adversely affect the results of the assay.

Also, while a cellular lawn is an extremely convenient way to screen large numbers of candidates, other techniques can also be used in accordance with the present invention. These techniques include the use of multiwell plates, and the various devices used for the preparation of the combinatorial library, such as pins, tea bags, etc. One may grow the cells separately in relation to the nature of the various devices, where the device may then be contacted with the cells or have the cells grown on the device. The device may be immersed in an appropriate culture, seeded with the cells, or otherwise provided for contact between the cells and the candidate agent. After adding the cytotoxic agent, one may then analyze for lysis in a variety of methods well-known in the art.

In addition, one may wish to know whether the agent has agonist or antagonist activity. The subject assay techniques provide for a rapid way for determining those agents present in the library which bind to the target protein. Once one has substantially narrowed the number of candidate agents, one can use more sophisticated assays for detecting the activity of the agent itself In this way, one can perform a rapid screen to determine binding affinity and specificity, followed by a more intensive screen to determine activity. Various techniques exist for determining activity, where the cells may be modified, so that a marker gene will be activated which will provide for a detectable signal. Conveniently, the signal may be associated with production of a dye, the production of a surface membrane protein which can be detected with labeled antibodies, or the secretion of a protein which can be detected in the supernatant by any of a variety of techniques. For example, the gene that is expressed may be luciferase modified to have a leader sequence so as to be secreted, whereby the supernatant can then be screened for light generation formation by using an appropriate substrate.

Various protocols may be employed for screening the library. To some degree, this will depend upon the nature of the preparation of the agents. For example, the agents may be bound to individual particles, pins, membranes, or the like, where each of the agents is segregatable. In addition, the amount of agent available will vary, depending upon the method employed for creating the library. Furthermore, depending upon the nature of the attachment of the agent to the support, one may be able to release aliquots of an agent, so as to carry out a series of assays. In addition, the manner in which the agents are assayed will be affected by the ability to identify the agent which is shown to have activity.

Where the agents are individually on a surface in a grid, so that at each site of the grid one knows what the composition is, one can provide a cellular lawn which is similarly organized as a grid and may be placed in registry with the agents bound to the solid surface. Once the lawn and solid substrate are in registry, one may release the agents from the surface in accordance with the manner in which the agents are attached. After sufficient time for the agents to bind to the proteins on the cellular surface, one may wash the cellular lawn to remove non-specifically bound agents. One or more washings may be involved, where the washings may provide for varying degrees of stringency, depending upon the desired degree of affinity. After the washings have been completed, mammalian blood or plasma may then be added and incubated for sufficient time for cytotoxicity. The plasma or blood may then be removed and plaques observed, where the nature of the agent can be determined by virtue of the position in the grid. The plasma or blood can be free of any components that would naturally kill the cells of the lawn.

Since the preparative process may be repeated, one could prepare a plurality of solid substrates, where the same agents are prepared at the comparable sites, so that the screening could be repeated with the same or different cells to determine the activity of the individual agents. In some instances, the identity of the agent can be determined by a nucleic acid tag, using the polymerase chain reaction for amplification of the tag. (See, e.g., PCT Application No. WO93/20242.) In this instance, the agents that are active may be determined by taking the lysate and introducing the lysate into a polymerase chain reaction medium comprising primers specific for the nucleic acid tag. Upon expansion, one can sequence the nucleic acid tag or determine its sequence by other means, which will direct the selection of the procedure is used to prepare the agent.

Alternatively, one may have tagged particles where the tags are releasable from the particle and provide a binary code that describes the synthetic procedure for the agents bound to the particle. (See, e.g., Ohlmeyer, et al. (1993) PNAS 90:10922.) These tags can conveniently be a homologous series of alkylene agents, which can be detected by gas chromatography-electron capture. Depending upon the nature of the linking group, one may provide for partial release from the particles, so that the particles may be used two or three times before identifying the particular agent.

While for the most part libraries have been discussed, any large group of agents can be screened analogously, so long as a BMP-1 related protein epitope or laminin 5 epitope can be joined to each of the agents. Thus, agents from different sources, both natural and synthetic, including macrolides, oligopeptides, ribonucleic acids, dendrimers, etc., may also be screened in an analogous manner.

The following examples explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### EXAMPLES

Unless otherwise stated, the following materials and methods were used in the examples of the present invention. The following GenBank Accession numbers are provided for BMP-1 related polynucleotides and polypeptides: human BMP-1 polynucleotide (GenBank Accession No. M22488); human BMP-1 polypeptide (GenBank Accession No. AAA51833); human mTld polynucleotide (GenBank Accession No. U91963); human mTld polypeptide (GenBank Accession No. AAB93878); human mTll-1 polynucleotide (GenBank Accession No. NM_{—}012464); human mTll-1 polypeptide (GenBank Accession No. NP_{—}036596); human mTll-2 polynucleotide (GenBank Accession No. NM_{—}012465); human mTll-2 polypeptide (GenBank Accession No. NP_{—}036597). Each of these GenBank references is incorporated herein by reference in its entirety.

### Example 1

### Assay for Laminin 5 Processing Activity

A comparative analysis of the abilities of BMP-1 and MMP-2 to process the α3 chain of laminin 5 was performed. Laminin 5 was obtained as described in Marinkovich et al., (1992) J. Biol. Chem. 267:17900-17906. A digestion of approximately 1 μg of laminin 5 with 30 ng/ml BMP-1 for 16 hours at 37° C. resulted in processing of the laminin α3 chain. FIG. 2 shows a western blot of these results, using an antibody to laminin 5 generated as described in Marinkovich et al., (1992) J. Biol. Chem. 267:17900-17906. FIG. 2, lane 1, is no-enzyme control. FIG. 2, lane 2, is BMP-1 addition as described above. The results show that laminin α3 chain was completely processed from 200 kDa to 165 kDa by BMP-1.

In contrast, no digestion of laminin 5 was seen with 100 ng/ml MMP-2 (over three times the amount of BMP-1 on a molar basis) at 37° C. for 16 hours. (FIG. 2, lane 3.) As a positive control, MMP-2 completely digested an equimolar quantity of type IV collagen under the same conditions (data not shown). These studies conflicted with prior reports that MMP-2 digests laminin 5. However, in the case of the previous studies, rat laminin 5 was used as the substrate. (Giannelli et al., supra.) The MMP-2 cleavage site in the human γ2 chain of laminin 5 is between the glycine and alanine of the sequence NCEHG/AFSCPACYN (SEQ ID NO:6), while that for the rat γ2 chain of laminin 5 is between the alanine and leucine of the sequence RTAAA/LTSCPACYN (SEQ ID NO:7). The sequence of rat laminin 5 at the MMP-2 cleavage site in the γ2 chain shows a complete lack of homology to the corresponding site the γ2 chain of human laminin 5 (underlined regions of above amino acid sequences, SEQ ID NO:6 and SEQ ID NO:7).

### Example 2

### Assay for Laminin 5 Processing Activity in Keratinocytes

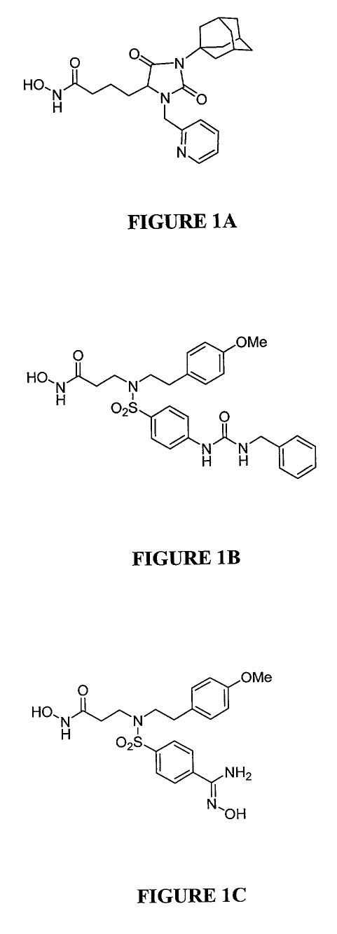

An analysis of BMP-1 processing on keratinocytes was performed. These studies utilized inhibitors of BMP-1 activity, specifically, Compound 1, Compound 2, and Compound 3. (FIGS. 1A, 1B, and 1C.) These inhibitory compounds showed a 500- to 1000-fold greater specificity of inhibition of BMP-1 than of other MMPs, including MMP-1, MMP-2, and MMP-9, in assays of collagen proteolysis. All inhibitors showed inhibition of BMP-1 activity. Compound 3 was slightly less potent than Compound 1 and Compound 2.

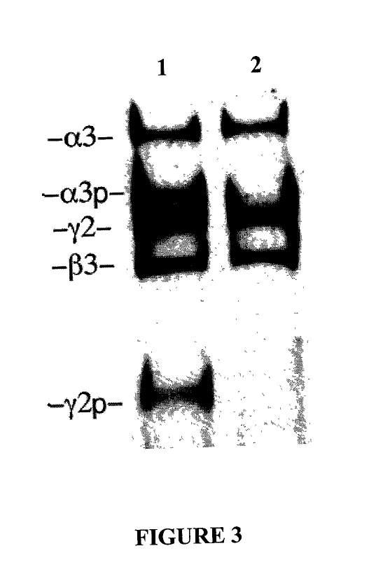

Each of the inhibitors was added to freshly plated cultures of normal human keratinocytes. After 16 hours, laminin 5 was extracted from cultures and analyzed by western blot using a polyclonal antibody to laminin 5. (Marinkovich et al., (1992) J. Biol. Chem. 267:17900-17906.) Results showed that BMP-1 inhibition with Compound 2 did not significantly change the level of expression of laminin 5, but completely inhibited processing of both the α3 and γ2 chains of laminin 5. (FIG. 3: lane 1, control; lane 2, Compound 2.) It should be noted that the BMP-1 related protein inhibitors used in these studies were shown to have no toxic effects on cells, even at 100 times the concentration which inhibited BMP-1 activity.

The human γ2 chain of laminin 5 has a BMP-1 processing site which is between glycine 434 and aspartic acid 435 in the sequence GDCYSG/DENPDTEC (SEQ ID NO:8). The human α3 chain of laminin 5 has a BMP-1 processing site which is between glycine and aspartic acid in the sequence GALQFG/DIPTSHLL (SEQ ID NO:9). Analysis of the γ2 chain cleavage sequence on human laminin 5 showed that the site has optimal sequence specificity for processing by BMP-1 related proteins. The intersection of the G3 and G4 domains of laminin 5 (the predicted sequence of α3 chain processing, based on electrophoretic migration of processed α3 chain) also shows an optimal sequence for cleavage by BMP-1 related proteins.

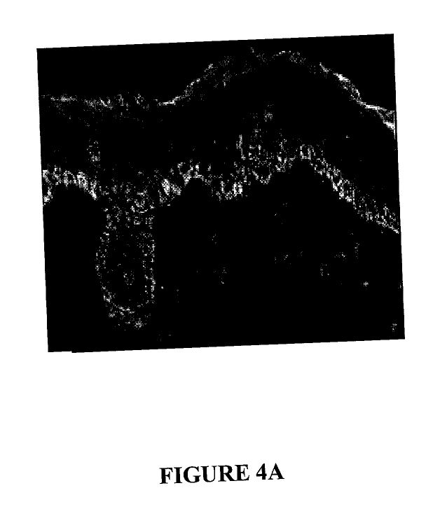

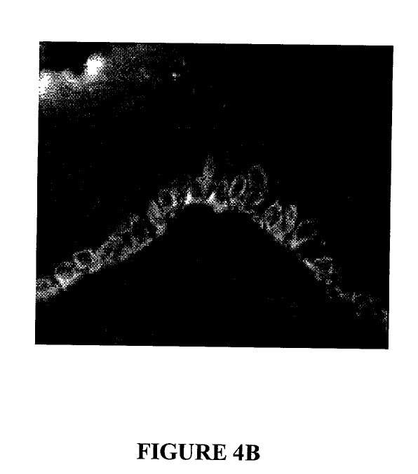

These results strongly implicated the BMP-1 related proteins in laminin γ2 chain and α3 chain processing in keratinocytes in vitro. Expression of BMP-1 in basal cells of the epidermis and the basal follicular epithelium, the same cell population that synthesizes laminin 5 is shown in FIG. 4A and FIG. 4B, respectively. The polyclonal antibody against human BMP-1 was generated in rabbits. Human BMP-1 cDNA was placed into an insect cell expression vector and used to express full-length human BMP-1 protein in drosophila cells. The BMP-1 protein was purified and used to immunize rabbits for the generation of polyclonal antibodies. The rabbit polyclonal antibody to human BMP-1 produced recognizes BMP-1, mTld, mTll-1, and mTll-2.

### Example 3

### Inhibition of Keratinocyte Migration

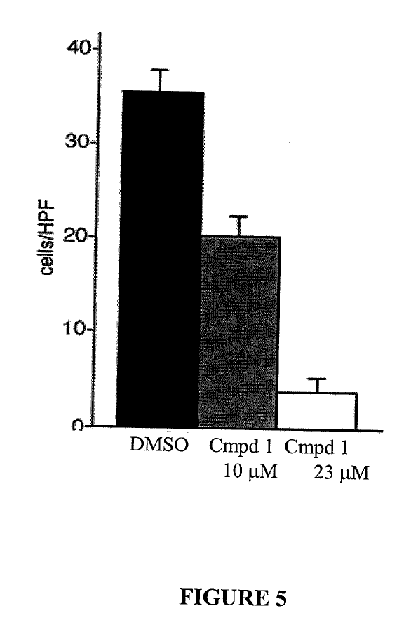

Compounds 1 and 2 were used to examine the functional effects of processing by BMP-1 related proteins on keratinocyte migration. Both compounds were found to potently inhibit keratinocyte migration. Keratinocytes were analyzed for migration in Boyden chambers in the presence of increasing amounts of the inhibitors. As shown in FIG. 5, the inhibition of migration of keratinocytes was dose dependent for Compound 1 (0 μM (DMSO control), 10 μM, and 23 μM). At optimal doses of either compound, greater than 80% inhibition of migration was observed. The dose required to achieve nearly complete inhibition of migration was 23 μM for Compound 1 and 10 μM for Compound 2. In comparison, previously described cyclic peptide-based inhibitors of MMP-2 and MMP-9 were much less specific and were able to achieve similar levels of inhibition of cell migration only at 500 μM concentrations. (Koivunen et al. (1999) Nature Biotechnology 17:768-774.)

### Example 4

### Processing of Laminin-5 by BMP-1 Related Proteins

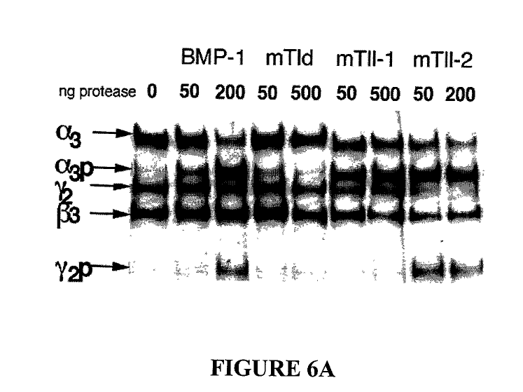

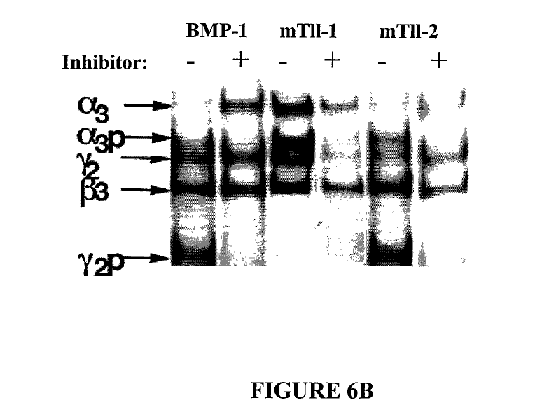

The processing of laminin 5 by various BMP-1 related proteins was examined. Unprocessed laminin 5 was deposited onto the surface of culture dishes by keratinocytes cultured in the presence of 10 μM Compound 2, which prevented processing of laminin 5. The cells were then removed from the culture dish by 20 mM ammonium sulfate, the dish was washed, and the matrix was incubated in increasing amounts of each BMP-1 related protein. After proteolytic digestion, the matrix was extracted and examined by western blot analysis. (FIG. 6A.) The results showed that BMP-1, mTld, mTll-1, and mTLL-2 processed laminin 5. As seen in this particular experiment (FIG. 6A), BMP-1, mTld, mTll-1, and mTLL-2 all cleaved the α3 chain of laminin 5. BMP-1 and mTLL-2 cleaved the γ2 chain of laminin 5. Additionally, mTll-2 showed more potent cleavage activity toward the α3 chain and γ2 chain of laminin 5 compared to the other BMP-1 related proteins.

Using the assay described above, the inhibitory activities of Compound 2 on BMP-1, mTld, mTll-1, and mTll-2 were examined. As shown in FIG. 6B, the inhibitor at 10 μM inhibited cleavage of laminin 5 γ2 chain by BMP-1 and mTLL-2, and inhibited cleavage of laminin 5 α3 chain by BMP-1, mTLL-1, and mTLL-2.

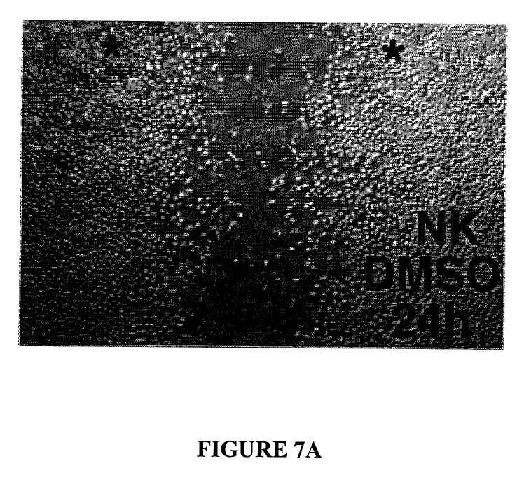

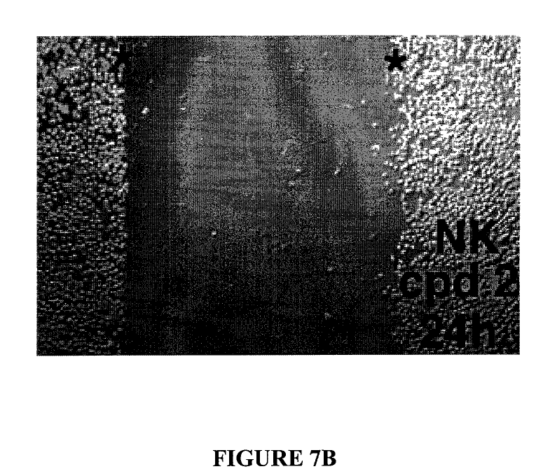

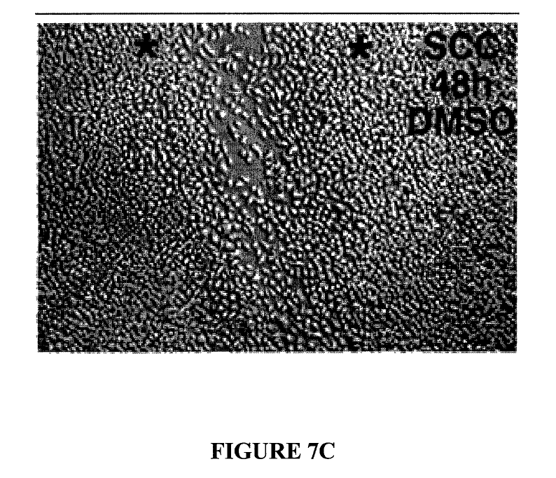

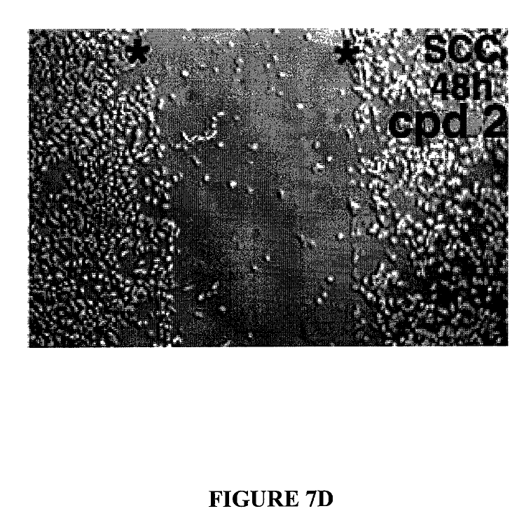

To approximate in vivo epidermal cell migration, during which epidermal cells move as sheets, scratch assays were utilized. Normal human keratinocytes were plated to confluence in the presence of either of the inhibitors (10 μM) or DMSO, and the cells allowed to attach for 6 hours. A 2 mm scratch was produced in the culture, marked, and the migration of the cells studied for the following 24 to 48 hours. As shown in FIG. 7A, control keratinocytes (NK) re-epithelialized a significant portion of the scratched area after 24 hours in the absence of the inhibitor (edges of original scratch are shown with asterisks). In contrast, keratinocytes treated with Compound 2 showed almost a complete lack of migration into the scratch after the same time period. (FIG. 7B.) When scratched plates were subsequently washed after 24 hours of incubation with the inhibitors, and incubated for an additional 24 hours in the absence of inhibitors, normal migration equivalent to that of the control conditions was noted.

In addition, squamous carcinoma cell line SCC-25 (ATCC Number CRL-1628) was examined by the described scratch assay using the inhibitors. As shown in FIG. 7C (control) and FIG. 7D (Compound 2), inhibition of BMP-1 related protein activity in SCC-25 cells treated with Compound 2 was effective at preventing SCC-25 migration. The inhibition of cell migration by Compound 2 was as effective in cells derived from squamous cell carcinoma as in normal keratinocytes, even after 48 hours (compare FIG. 7B, normal keratinocytes, to FIG. 7D, squamous cell carcinoma cells). Washing out of the inhibitors for an additional 48 hour time period restored normal SCC migration.

### Example 5

### Dose Dependent Inhibition of Keratinocyte and SCC Cell Migration

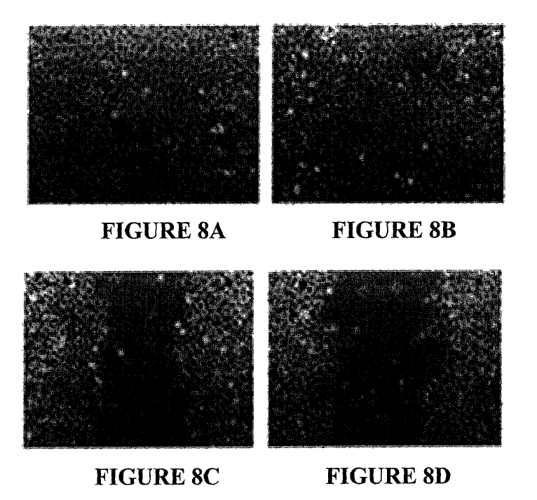

A dose-dependent study of Compound 2 was performed on keratinocyte cultures. In these assays, a scratch was placed in a confluent layer of cells, and the migration of the cells into the scratched area was assessed after 48 hours. In the absence of inhibitor, normal immortalized keratinocytes (NIK) migrated into the scratch area. (FIG. 8A.) In the presence of Compound 2 (FIG. 8B, 1 μM; FIG. 8C, 5 μM; FIG. 8D, 8 μM), a dose-dependent inhibition of migration of NIK into the scratch area was observed.

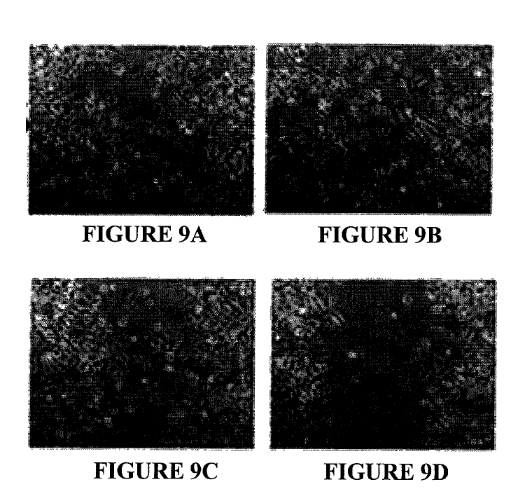

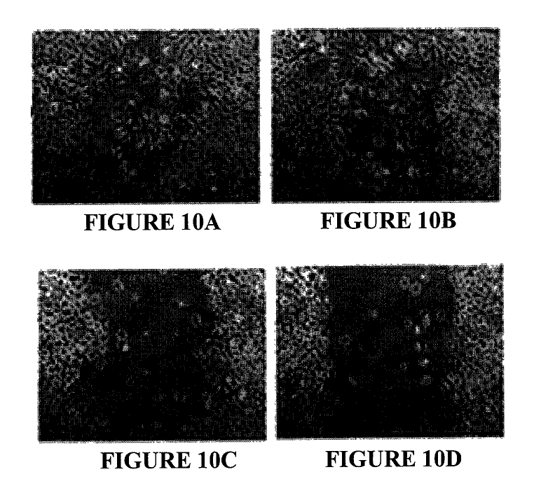

Similar results were obtained with squamous cell carcinoma cells lines. FIGS. 9A through 9D show the dose-dependent effect of Compound 2 on migration of squamous cell carcinoma cell line SCC-15 into the scratch area. (FIG. 9A, control; FIG. 9B, 1 μM; FIG. 9C, 5 μM; FIG. 9D, 10 μM.) FIGS. 10A to 10D show the dose-dependent effect of Compound 2 on migration of squamous cell carcinoma cell line SCC-25 into the scratch area. (FIG. 10A, control; FIG. 10B, 1 μM; FIG. 10C, 5 μM; FIG. 10D, 10 μM.)

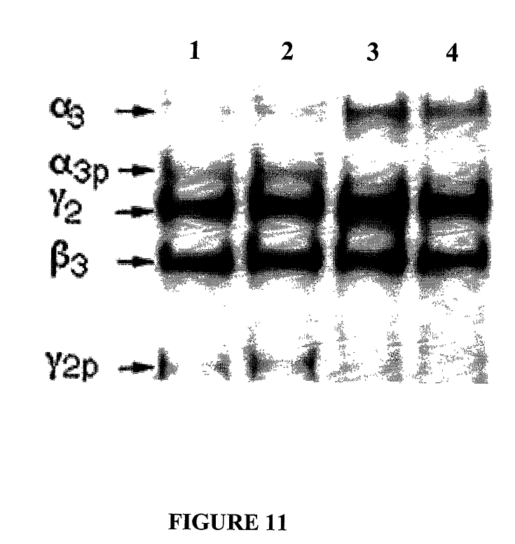

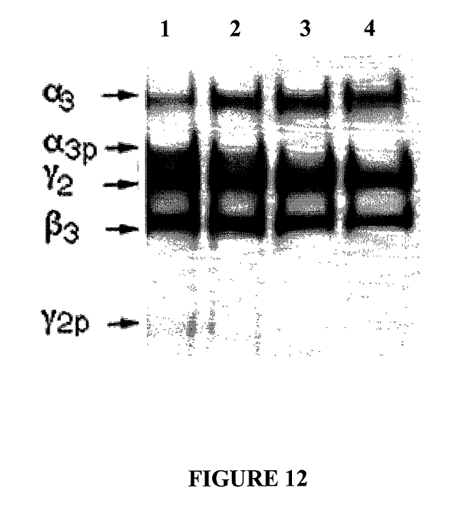

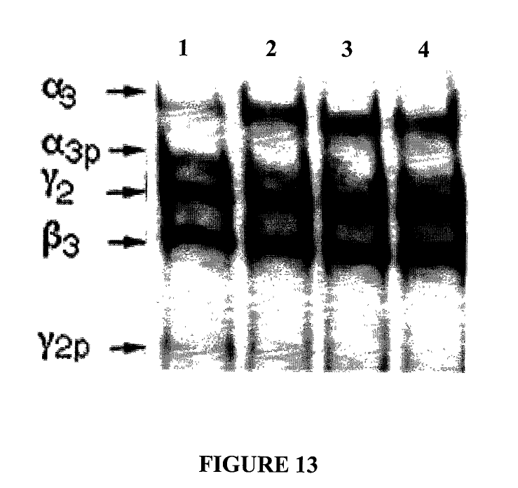

The dose-dependent inhibition of migration of normal immortalized keratinocytes (NIK), SCC-15 cells (ATCC Number CRL-1623), and SCC-25 cells correlated with the inhibition of laminin 5 processing. This is shown by western blot analysis. FIG. 11 shows the dose-dependent effect of Compound 2 on processing of laminin 5 in normal immortalized keratinocytes after 48 hours. (FIG. 11: lane 1, control; lane 2, 1 μM; lane 3, 5 μM; lane 4, 8 μM.) FIG. 12 shows the dose-dependent effect of Compound 2 on processing of laminin 5 in squamous cell carcinoma cell line SSC-15 after 48 hours. (FIG. 12, lane 1, control; lane 2, 1 μM; lane 3, 5 μM; lane 4, 10 μM.) FIG. 13 shows the dose-dependent effect of Compound 2 on processing of laminin 5 in squamous cell carcinoma cell line SSC-25 after 48 hours. (FIG. 13: lane 1, control; lane 2, 1 μM; lane 3, 5 μM; lane 4, 10 μM.)

### Example 6

### Laminin 5 Specificity

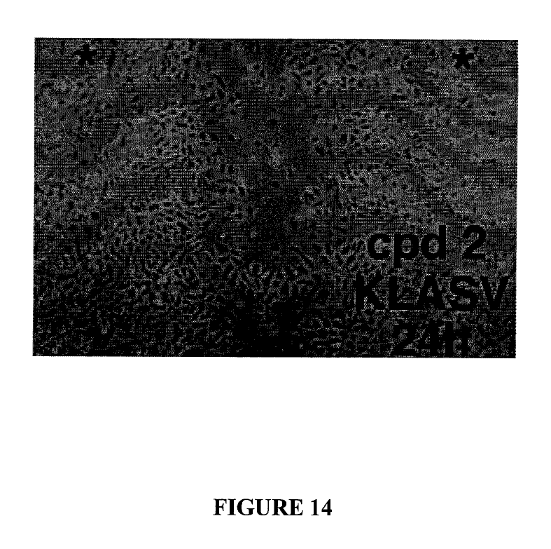

To determine whether the effect of inhibitory Compounds 1, 2, and 3 on cell migration was specific for laminin 5, scratch assays were performed in the presence of the inhibitors using KLASV cells, a keratinocyte cell line derived from a patient with epidermolysis bullosa. KLASV cells do not synthesize laminin 5. To allow for attachment to the tissue culture dish, KLASV cells were plated in tissue culture wells previously coated with type I collagen. In the presence of Compound 2, KLASV cells showed significant migration into the scratch after 24 hours (FIG. 14), comparable to non-treated control cultures. Similar results were seen when using Compound 3 as an inhibitor. These results suggested that the effect of BMP-1 related protein inhibition on cell migration was specific to inhibition of laminin 5 processing.

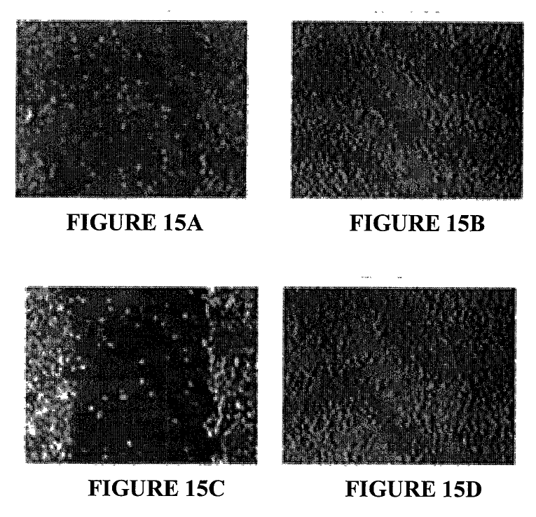

The specificity of the inhibitors was further assessed by comparison of the efficacy of Compound 2 on inhibiting keratinocyte and SCC cell migration on type I collagen coated dishes compared to non-coated dishes. Compound 2 (10 μM) was equally effective at inhibiting cell migration, regardless of whether an alternative collagen substrate was provided to the cells. Results using KLASV cells plated onto type I collagen coated dishes are shown. (FIG. 15A, 0 hr control; FIG. 15B, 48 hr control; FIG. 15C, 0 hr, Compound 2; FIG. 15D, 48 hr, Compound 2.) The inhibition of cell migration appeared to be dependent on laminin 5 production, as migration of the keratinocyte cell line KLASV, deficient in laminin 5 production, was not inhibited by Compound 2.

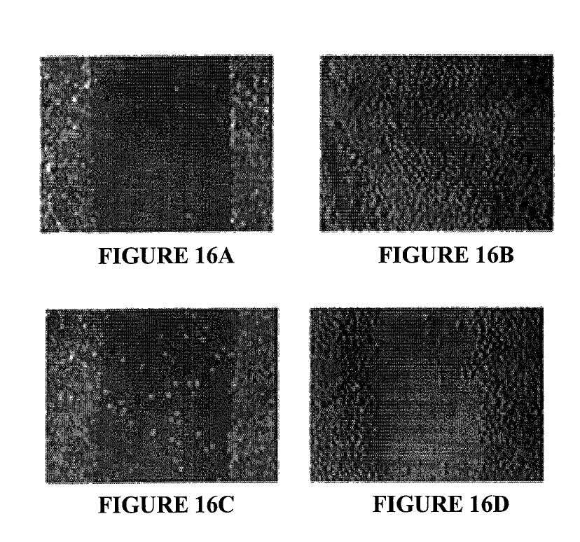

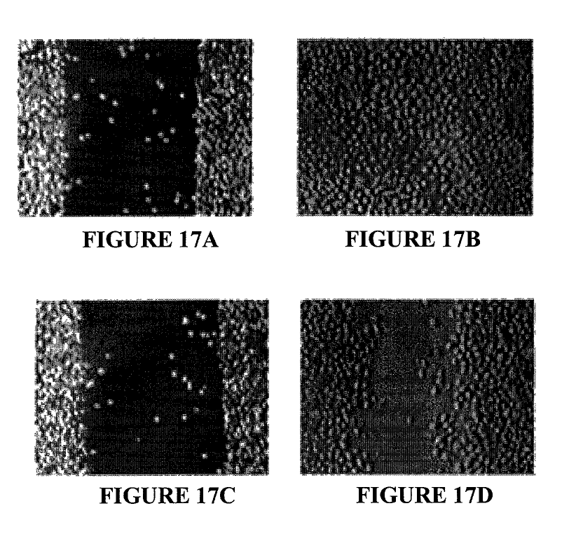

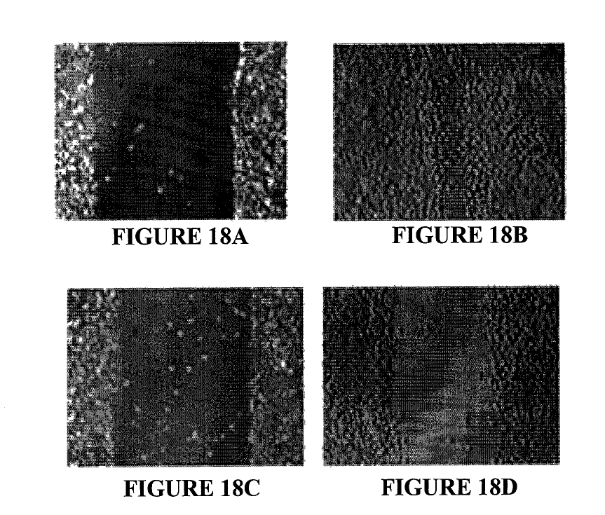

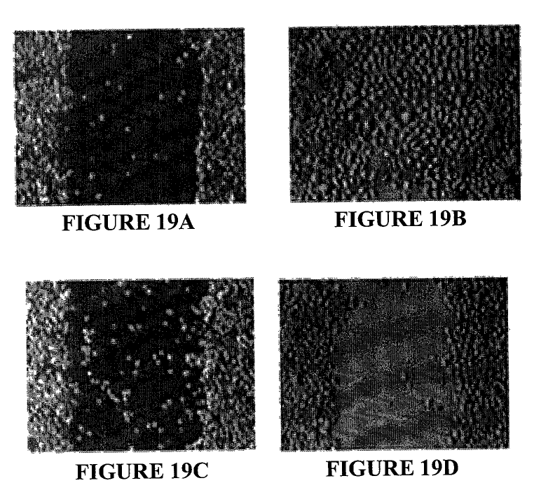

Results using normal immortalized keratinocytes (NIK) plated onto type I collagen coated dishes are shown. (FIG. 16A, 0 hr control; FIG. 16B, 48 hr control; FIG. 16C, 0 hr, Compound 2; FIG. 16D, 48 hr, Compound 2.) Results using squamous cell carcinoma cell line SCC-25 plated onto type I collagen dishes are shown. (FIG. 17A, 0 hr control; FIG. 17B, 48 hr control; FIG. 17C, 0 hr, Compound 2; FIG. 17D, 48 hr, Compound 2.) Results using normal immortalized keratinocytes (NIK) plated onto plastic are shown. (FIG. 18A, 0 hr control; FIG. 18B, 48 hr control; FIG. 18C, 0 hr, Compound 2; FIG. 18D, 48 hr, Compound 2.) Results using squamous cell carcinoma cell line SCC-25 plated onto plastic are shown. (FIG. 19A, 0 hr control; FIG. 19B, 48 hr control; FIG. 19C, 0 hr, Compound 2; FIG. 19D, 48 hr, Compound 2.)

These results showed that providing cells with a collagen substrate was not sufficient for migration. Production of laminin 5, and subsequent processing of laminin 5 by BMP-1 related proteins, was required for cell migration to occur. Keratinocytes and squamous carcinoma cells were not able to migrate if laminin 5 processing was inhibited, even under conditions where the cells were plated on a collagen substrate. These results suggested that the deposition of laminin 5 onto the culture substrate, coupled with an inability to process it, results in inhibition of cell migration. In summary, these results demonstrate that processing of laminin 5 by BMP-1 related proteins has a profound effect on the control of normal keratinocyte and squamous carcinoma cell migration.

### Example 7

### Keratinocyte Attachment Studies

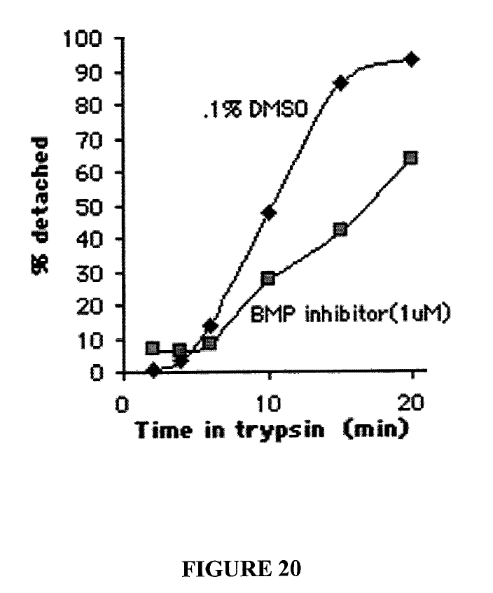

The mode of attachment of keratinocytes to processed and unprocessed laminin 5 was examined by two methods. First, normal keratinocytes were cultured for 24 hours in the presence or absence of Compound 2, were incubated in 0.05% trypsin, and the percentage of cells which detached at the time intervals indicated was determined. Cells treated with Compound 2 were more resistant to trypsin treatment compared to untreated cells, suggesting that they were more firmly attached to substrate. (FIG. 20.)

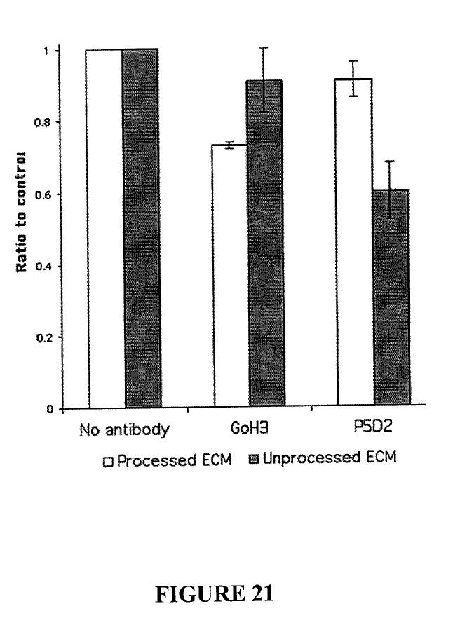

Keratinocytes were cultured in the presence or absence of the inhibitors, and then removed to produce matrices containing either processed or unprocessed laminin 5, as shown in FIG. 21. Normal keratinocytes in the absence of inhibitor were examined for attachment to processed versus unprocessed laminin 5 matrices in the presence of inhibitory antibodies to α6β4 integrin (GoH3) or to α3β1 integrin (P5D2) (commercially available antibodies to the integrins were obtained from Iowa Hybridoma Cell Bank). α6β4 integrin inhibition significantly inhibited keratinocyte attachment to unprocessed but not processed laminin 5. Conversely, α3β1 integrin inhibition significantly inhibited attachment to unprocessed but not processed laminin 5.

These results suggested processing of laminin 5 alters the mechanisms by which cells interacted with laminin 5. The results indicated that α6β4 integrin may preferentially bind to processed laminin 5, whereas α3β1 integrin may preferentially bind to unprocessed laminin 5.

### Example 8

### Effect of BMP-1 Inhibitors on in Vitro SCC Invasion

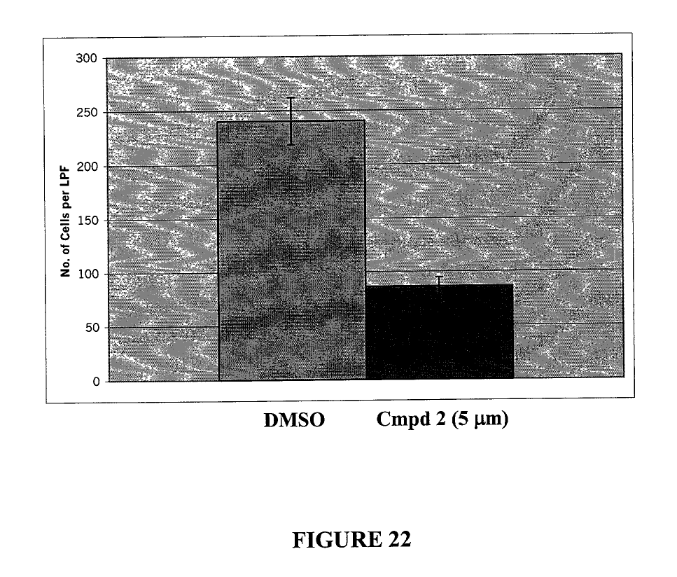

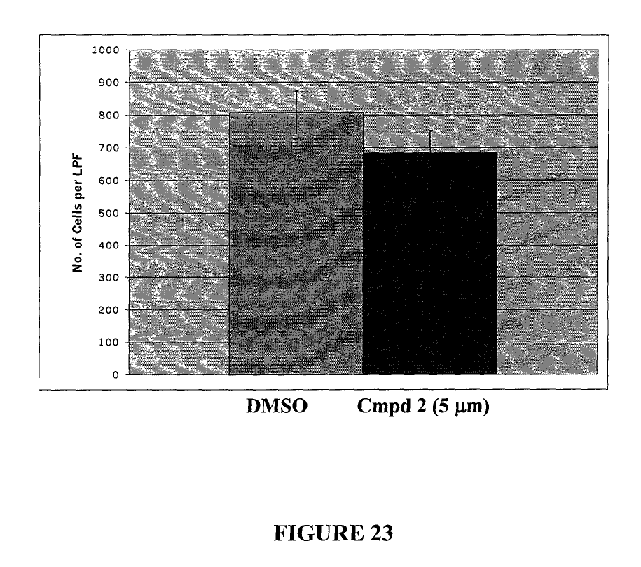

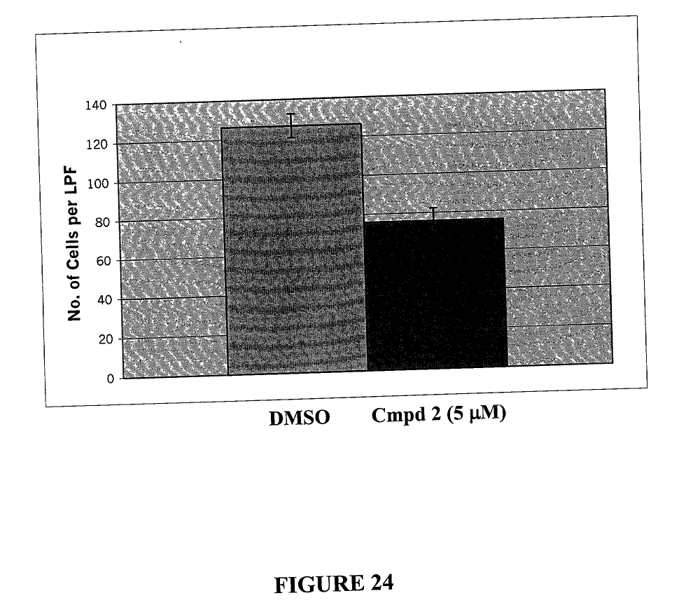

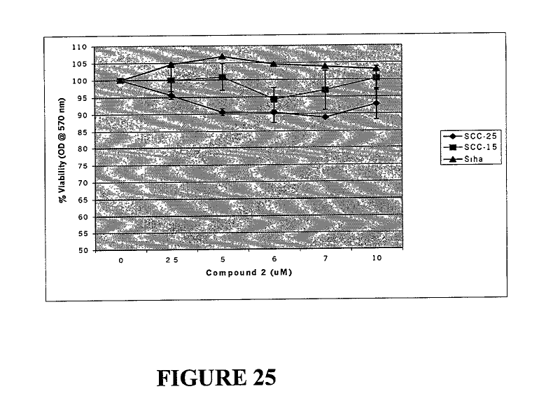

The effects of compound 2 on the invasion of SCC cells were examined using the BD BIOCOAT MATRIGEL INVASION CHAMBERS in vitro system (Becton Dickinson; Franklin Lakes, N.J.) with standard protocols. The assay is a standard in vitro measure of tumor cell invasion. Tumor cells are plated atop a layer of fabricated basement membrane and allowed to migrate over the course of 24 hours towards conditioned 3T3 medium, which contains a chemo-attractant, located in a lower chamber. As shown, Compound 2 (5 μM) was able to inhibit the invasion SCC-25 cells (FIG. 22) and SCC-15 cells (FIG. 24). As shown in FIG. 23, Compound 2 was not able to inhibit the invasion of SiHa cells, an SCC cell line which has lost the ability to produce laminin 5 (ATCC Number HTB-35). An MTT toxicity assay indicated that each cell line showed little or no toxicity in a 24 hour time period, suggesting that the effect was specific to laminin 5 processing inhibition. (FIG. 25.)

### Example 9

### Overexpression of BMP-1, mTld, mTll-1, and mTll-2 in Keratinocytes, SCC Cells and Tumors

In order to observe the in vitro effects of the over expression of BMP-1 and/or related proteins, and to compare these with the results of in vitro inhibition described in the preliminary studies, full length cDNAs coding for BMP-1 and/or BMP-1 related proteins are overexpressed using pCEP mammalian expression vectors. These are transfected into SCC-25 cells using lipofectamine. After selection of transfected cells with puromycin, laminin 5 is extracted from medium and cell fractions. These fractions are analyzed by western analysis using laminin 5 and pAbs to verify increased processing of laminin 5 and increased secretion of BMP-1 and/or BMP-1 related proteins into culture medium of transfected cells, compared to vector only controls. Transfected cells are assessed by phase contrast microscopy to look for alterations of cell morphology and dual label IDIF microscopy to evaluate the colocalization of laminin 5 with integrins and other basement membrane zone (BMZ) components. Cell migration is evaluated, for example, through use of the Boyden chamber and scratch assays.

### Example 10

### In vivo Analysis of Overexpression of BMP-1, mTld, mTll-1, and mTll-2

Inhibition and overexpression of BMP-1 and/or BMP-1 related proteins in vivo is analyzed in tumors. For overexpression studies, cDNA of BMP-1 and/or BMP-1 related proteins are cloned into a modified Lazarus-family virus (i.e., vector), which has blasticidin resistance connected by an internal ribozyme entry sequence (IRES), which allows two genes to be engineered into one virus vector. The Lazarus-family of virus vectors provides efficient transfection into epithelial cells and squamous cells, and squamous cell carcinoma cells, as well as long-term expression of transfected genes. SCC-25 cells are transduced with retroviral vector containing the cDNA or vector alone. Transduced cells (5×10⁵ in 200 μl sterile PBS) are injected into nude mouse skin in fractionated amounts of four injections of 50 μl (SCC-25 have been demonstrated to form tumors in nude mice, see, e.g., ATCC catalog). Animals are monitored three times a week for the development of tumors, and evidence of tumor formation is expected 3 to 4 weeks post-injection. When tumors reach 1 cm in size, or in six weeks, the mice are sacrificed, and the tumors are measured, weighed, and examined by routine histology. Immunohistochemistry and immunofluorescence is carried out using a panel of integrin, laminin 5, and other antibodies.

### Example 11

### In vivo Analysis of Inhibition of BMP-1, mTld, mTll-1, and mTll-2

To assess inhibition of BMP-1 related proteins in tumors, BMP-1 inhibitors are introduced into an animal model system. The inhibitors are injected intradermally in the area of the induced wound or in the area of injected tumor cells. The BMP-1 inhibitors are used in injections of about 50 μl with stock solutions ranging up to about 2 mM. DMSO containing PBS is used as control. Initial studies involve harvesting the tissue surrounding the 4-mm induced wound or the tumor injection site using a 6-mm full thickness skin punch at 2 hour intervals up to twelve hours post-injection. These biopsies are assayed for concentrations of the inhibitor.

Once the half-lives of the inhibitors are determined at the tumor site, and the intervals of injection are determined so that a relatively steady 1 μM to 2 μM concentration of the inhibitor is obtained, the appropriate concentration and intervals is used to test the in vivo inhibition of induced wounds and SCC tumors. Tumor growth and reepithelialization is studied as above. Techniques utilizing controlled release of biodegradable materials are tested. (Egilmez et al. (1998) Canc. Immuno. Immunother. 46:21-24; Mathiowitz et al. (1997) Nature 386:410-414.) In addition, intraperitoneal injection of the inhibitor is tested.

### Example 12

### Inhibition of Metastasis

A tumor metastasis model is utilized to test the ability of BMP-1 inhibitors to reduce metastasis of SCC-25 cells injected via tail vein to nude mice. Initially, inhibitors will be injected intraperitoneally, using concentrations of 2 mM and volumes of 250 μl. Blood is obtained from tail vein bleeds at 2 hour intervals following injection up to 12 hours and is assayed for inhibition of BMP-1 activity in order to determine the level of circulating inhibitor. The ultimate goals of these studies is to determine a concentration and interval of injection to obtain a steady state concentration of 1 μM to 2 μM of the inhibitors. Mice are monitored closely throughout these experiments for potential signs of toxicity, and following these studies, are sacrificed. Autopsies are performed to determine any toxic effects of the inhibitor. Following these control experiments, nude mice are injected with sufficient inhibitor to maintain a steady state level of 1 μM to 2 μM of each of the inhibitors. This is followed by injection in the tail vein with 500 SCC-25 cells in 50 μl of PBS. Inhibitor levels will be maintained for one week in the injected mice (DMSO controls are used in parallel). Inhibitor injections are stopped and the mice are sacrificed three weeks later and autopsied. Lungs are serially sectioned and assayed for number of tumor metastases. A continuing parenteral infusion of the inhibitor delivered by a mini pump can be utilized to maintain a 1 μM to 2 μM steady state level.

### Example 13

### Inhibition of SCC Tumor Invasion

A model of SCC tumor invasion in vivo is utilized to test the ability of topically applied BMP-1 inhibitors to reduce invasion of SCC cells into the dermis of nude mice. A 1 cm² section of full thickness skin is excised from the dorsal-lateral trunk of each nude mouse. A 1 cm² square section of devitalized porcine dermis containing human SCC cells are sutured in place into the surgically created space. A gel formulation of BMP-1 inhibitor (0.5 ml), able to penetrate through the epidermis to the dermal layer, is placed into the graft site. In a parallel group of control mice, the vehicle is applied to the wound without inhibitor. At two day intervals after grafting, additional inhibitor gel or vehicle gel without inhibitor is applied to the graft site. At each two day interval, animals are inspected for any signs of morbidity and any animals showing signs of morbidity will be sacrificed by CO₂ inhalation. The grafts are kept in place for 3-4 weeks, and the animals are then sacrificed and the graft sites removed and examined. The grafts are examined by necroscopy to determine the presence of any metastases.

Various modifications and variations of the described methods and systems of the invention will be apparent to those skilled in the art without departing from the spirit and scope of the invention. Although the invention has been described in connection with specific preferred embodiments, it should be understood that the invention as claimed should not be unduly limited to such specific embodiments. Various modifications of the described modes for carrying out the invention which are obvious to those skilled in the present art and related fields are intended to be within the scope of the following claims. All references cited herein are incorporated by reference herein in their entirety.

What is claimed is:

1. A method of treating a condition characterized by increased expression or activity of laminin 5, the method comprising administering to a subject in need an effective amount of an agent that affects processing of laminin 5 by a BMP-1 related protein.

2. A method of treating cancer, the method comprising administering to a subject in need of an effective amount of an agent that affects processing of laminin 5 by a BMP-1 related protein.

3. A method of treating glioma, the method comprising administering to a subject in need an effective amount of an agent that affects processing of laminin 5 by a BMP-1 related protein.

4. A method of treating a condition characterized by a neoplastic epithelial cells, the method comprising administering to a subject in need an effective amount of an agent that affects processing of laminin 5 by a BMP-1 related protein.

5. The method of claim 4, wherein the epithelial cells are selected from the group consisting of squamous cells, keratinocytes, mucosal epithelial cells, gastrointestinal epithelial cells, corneal epithelia of the eye, and epithelial cells of the urinary and reproductive tract.

6. A method of treating squamous cell carcinoma, the method comprising administering to a subject in need an effective amount of an agent that affects processing of laminin 5 by a BMP-1 related protein.

7. The method of claim 6, wherein the squamous cell carcinoma is selected from the group consisting of skin cancer, lung cancer, head cancer, neck cancer, oral cancer, cervical cancer, tongue cancer, gastric cancer, colorectal, throat cancer, cancer of the urinary tract, cancer of the reproductive tract, esophageal cancer, throat cancer, and bronchiogenic carcinoma.

8. The method of claim 6, wherein the agent affects processing of the α3 chain of laminin 5.

9. The method of claim 6, wherein the agent affects processing of the γ2 chain of laminin 5.

10. The method of claim 6, wherein the agent affects processing of laminin 5 by BMP-1.

11. The method of claim 6, wherein the agent affects processing of laminin 5 by mTld.

12. The method of claim 6, wherein the agent affects processing of laminin 5 by mTll-1.

13. The method of claim 6, wherein the agent affects processing of laminin 5 by mTll-2.

14. The method of claim 6, wherein the agent additionally confers an anti-scarring effect.

15. A composition for the treatment of a condition associated with increased expression or activity of laminin 5, the composition comprising an agent that affects processing of laminin 5 by a BMP-1 related protein and an acceptable carrier.

16. A composition for the treatment of squamous cell carcinoma, the composition comprising an agent that affects processing of laminin 5 by a BMP-1 related protein and an acceptable carrier.

17. The composition of claim 16, wherein the agent affects at least one protein selected from the group consisting of BMP-1, mTld, mTll-1, and mTll-2.

18. A method of diagnosing the presence of a condition characterized by increased expression or activity of laminin 5 in a subject, the method comprising:
(a) obtaining a sample;
(b) detecting the level of expression or activity of a BMP-1 related protein in the sample;
(c) comparing the level of expression or activity of the BMP-1 related protein in the sample to a standard level of expression or activity of the BMP-1 related protein.

19. A method of diagnosing the presence of squamous cell carcinoma in a subject, the method comprising:
(a) obtaining a sample;
(b) detecting the level of expression of a BMP-1 related protein in the sample;
(c) comparing the level of expression or activity of the BMP-1 related protein to a standard level of expression or activity of the BMP-1 related protein.

20. The method of claim 19, wherein the BMP-1 related protein is selected from the group consisting of BMP-1, mTld, mTll-1, and mTll-2.

21. The method of claim 19, wherein the sample is a tissue sample.

22. The method of claim 19, wherein the sample is a urine sample.

23. The method of claim 19, wherein the sample is a serum sample.

24. The method of claim 19, wherein the sample is a blood sample.

25. A diagnostic kit for use in diagnosing the presence of a condition associated with increased expression or activity of laminin 5 in a sample from a subject, the kit comprising:
(a) an anti-BMP-1 antibody reactive with BMP-1 related proteins; and
(b) a labeled reagent capable of forming a complex with a BMP-1 related protein or with the anti-BMP-1 antibody.

26. A method of screening for an agent that affects the processing of laminin 5 by BMP-1 related proteins, the method comprising:
(a) contacting a sample containing unprocessed laminin 5 with at least one BMP related protein and the agent;
(b) measuring the level of processed laminin 5 in the sample;
(c) measuring the level of processed laminin 5 in a control sample; and
(d) comparing the level of processed laminin 5 in the sample with the level of processed laminin 5 in the control sample.

27. An isolated polypeptide comprising a BMP-1 cleavage sequence, the polypeptide comprising the amino acid sequence of SEQ ID NO: 1.

28. An isolated polynucleotide encoding the polypeptide of claim 27.

29. An isolated polynucleotide that is complementary to the polynucleotide of claim 28.

30. An antibody that binds to the polypeptide of claim 27.

31. A method of screening for an agent that affects the processing of laminin 5 by BMP-1 related proteins, the method comprising:
(a) contacting a sample containing at least one polypeptide comprising SEQ ID NO: 1 with a BMP-1 related protein and an agent;
(b) measuring the level of the polypeptide that is processed;
(c) measuring the level of the polypeptide that is processed in a control sample; and
(d) comparing the level of the polypeptide that is processed in the sample to the level of polypeptide that is processed in the control sample.

32. An isolated polypeptide comprising a BMP-1 cleavage sequence, the polypeptide comprising the amino acid sequence of SEQ ID NO:2.

33. An isolated polypeptide comprising a BMP-1 cleavage sequence, the polypeptide comprising the amino acid sequence of SEQ ID NO:3.

34. An isolated polypeptide comprising a BMP-1 cleavage sequence, the polypeptide comprising the amino acid sequence of SEQ ID NO:4.

35. An isolated polypeptide comprising a BMP-1 cleavage sequence, the polypeptide comprising the amino acid sequence of SEQ ID NO:5.

36. A method of affecting laminin 5 expression or activity, the method comprising contacting laminin 5 with an effective amount of an agent that affects processing of laminin 5 by a BMP-1 related protein.
